(19) United States
(12) Patent Application Publication
(10) Pub. No.: US 2012/0015365 A1
(43) Pub. Date: Jan. 19, 2012

(54) Methods for Testing the Accuracy and Thermal Reliability of a PCR Thermal Cycler, and a Means for Implementing Said Methods

(76) Inventors: Anthony Bodin, Chatellerault (FR); Marc Priel, Levallois (FR); Pierre-Olivier Fraisse, Limeil-Brevannes (FR); Carole Couture, Bordeaux (FR); Cyril Falentin, Mordelles (FR)

(21) Appl. No.: 13/148,284

(22) PCT Filed: Feb. 11, 2010
(86) PCT No.: PCT/EP10/51753

§ 371 Date: Oct. 4, 2011

(30) Foreign Application Priority Data
Feb. 11, 2009 (FR) 0950860

(51) Int. Cl. C12Q 1/68; G01K 15/00

(52) U.S. Cl. 435/6.12; 374/1; 374/E15.001

(57) ABSTRACT

The present invention comprises a method for testing the accuracy and the reliability of a thermal unit of a thermal cycler or of an enclosure of a PCR thermal cycler thermally controlled by pulsed air, said thermal unit and enclosure thermally controlled by pulsed air comprising a plurality of locations for reaction mixture tubes, said method comprising the following steps:
a) arranging a set of temperature-measuring sensors in locations for reaction mixture tubes selected in said thermal unit and enclosure thermally controlled by pulsed air, said temperature-measuring sensors comprising:
   (i) a reaction tube suited to the type of thermal unit tested,
   (ii) a selected volume of liquid at least partially filling said reaction tube,
   (iii) a thermal probe at least partially submerged in said liquid, and
   (iv) at least one means for linking said thermal probe to a means for receiving a signal generated by said thermal probe,
said locations in which said temperature-measuring sensors are arranged being distributed across the surface of said thermal unit such that at least two temperature-measuring sensors are arranged in each of the thermal elements included in said thermal unit, or for the enclosures thermally controlled by pulsed air at the locations provided for the PCR tubes or capillary tubes such that at least the half of these locations are occupied by measuring sensors, and
b) starting the thermal cycler, under normal usage conditions, for at least one cycle programmed with temperature set values for a PCR reaction, and
c) at the same time as step b), measuring the temperature values with each of the measuring sensors, at a plurality of given moments.

### FIELD OF THE INVENTION

The present invention relates to the field of thermal calibration for thermal cycler apparatuses for performing PCR reactions, through physical means or biological means.

### PRIOR ART

The technique for amplifying nucleic acids through a polymerase chain reaction (PCR) is a method which use is so to speak a key element in the field of molecular genetics, including in fields as diverse as medical diagnosis, DNA analysis, in the medicolegal field, the detection of microorganisms that are pathogenic for humans, animals or plants, molecular biology research, and so on.

The PCR reaction consists in an in vitro enzymatic thermo-regulated reaction which amplifies the number of copies of a nucleic acid initially present in a sample. As is well known, the PCR reaction implements a reaction mixture comprising nucleic acids primers and thermo-resistant DNA polymerase, said reaction mixture being submitted to multistage thermoregulated reaction cycles. Classically, a PCR reaction cycle comprises successively (i) a step of denaturation of double-stranded DNA molecules performed at a specific temperature, close to 95° C., (ii) a step of hybridization of the nucleotide primers performed at a specific temperature, close to 60° C., then (iii) an elongation step performed at a specific temperature, close to 72° C.

PCR reactions are performed in specifically conceived apparatuses that are called thermal cyclers. Thermal cyclers are fitted with means for precisely regulating the temperature of the reaction mixture over time, in each of the hereabove mentioned steps (i), (ii) and (ii), for each PCR amplification cycle included in the test being conducted. One of the major component of a thermal cycler consists in a thermal unit, composed of several elements that are thermally regulated independently from each other, comprising a plurality of locations or [symbol] wells [symbol] intended to house reaction mixture tubes. As is well known, very precisely controlling the reaction medium temperature for the total duration of a PCR reaction is crucial to obtain reliable and reproducible results.

For example, a negative temperature drift, as compared to the selected temperature, during the step of denaturation may cause a DNA incomplete denaturation and/or cause an at least partial degradation of thermo-resistant DNA polymerase.

Furthermore, a negative temperature drift during the step of hybridization may cause a mismatch, including a non specific hybridization, of the nucleotide primers in the DNA to be amplified, which is susceptible to lead to false positive results. Conversely, a positive temperature drift during the elongation step may result in the absence of matching between primers and target DNA, which might produce false negative results.

In addition, a positive or negative temperature drift during the elongation step may cause the catalytic activity of the DNA polymerase to be reduced and the PCR reaction yield to be reduced accordingly.

As has been previously illustrated, the reproducibility of the thermal conditions under which PCR reactions are carried out is crucial for the biological relevance of the results of the effected tests, and the possibility to compare the results of distinct tests.

Many authors observed that the accuracy and the thermal reliability of PCR thermal cyclers (the expression [symbol] thermal regulation [symbol] of the PCR thermal cyclers may also be used, although it is less suitable and not so precise), as accurate they may be, is sufficiently variable to cause a defect in the reproducibility of the results obtained:
(i) for the same test on distinct thermal cyclers, including distinct thermal cyclers of the same commercial model,
(ii) between tests effected temporally successively, on a same thermal cycler, and even
(iii) between tests effected temporally simultaneously on the same thermal cycler, in reaction tubes arranged to be geographically distant from the surface of the thermal unit.

Such accuracy and thermal reliability heterogeneity amongst the reaction mixtures used during the PCR reaction, which may cause false positive or false negative results to be obtained, depending on the thermal drift types, is a matter of concern notably in the field of tests dedicated to medical diagnosis for diseases.

In order to overcome these technical drawbacks, devices for measuring the accuracy and the thermal reliability of PCR thermal cyclers have been described, which are capable of detecting local temperature variabilities in receiving wells of the thermal unit reaction tubes. The possible detection of this thermal variability in given locations of a thermal unit makes it possible to perform an accurate control operation of the accuracy and the thermal reliability of the thermal cycler, or a thermal [symbol] adjustment [symbol] of the thermal cycler (the expression thermal [symbol] calibration [symbol] of the thermal cycler may also be used).

Thermal adjustment operations in the thermal cyclers in every instance become necessary for laboratories using thermal cyclers and awaiting official certifications from government agencies.

One known type of device for measuring the accuracy and the thermal reliability of PCR thermal cyclers is the MTAS® system marketed by the Quanta Biotech company. This system consists in a plane support, with a surface similar to that of a thermal unit in a thermal cycler, onto which one or more thermal probes may be fixed, depending on the user choice. The support fitted with the one or more probes is placed on the upper part of the thermal unit, the one or more probes being then introduced into the corresponding wells of the thermal unit. Thereafter, the thermal cycler is started up with a predetermined program, notably for the temperature set values in the various steps of an amplification cycle and for temperature rise times or temperature reduction times up to the set values. Simultaneously, the signal generated by each of the thermal probes of the MTAS® device is transmitted to an electronic unit which records the evolution of the temperature values over time, in each well of the thermal unit in which a probe has been introduced. The measurement results enable to locate thermal regulation abnormalities of the tested thermal cycler and (i) either to simply take such abnormalities into consideration upon subsequent implementation of PCR reactions, (ii) or to make a fine adjustment of the thermal regulation system of the thermal cycler, that is to say to make an [symbol] adjustment [symbol] of the same. Another very similar measuring system is marketed under the trade names DRIFTCON® and DRIFTCON® RF by the eponymous company. This system differs from the previous system especially through the transmission of the data generated by the thermal probes through air rather than through a wire.

Another measuring system, based on the same principle as the one described hereabove, is composed of a plurality of thermal probes which may be introduced into the wells of a microtest plate of a type suited to the tested thermal cycler. The microtest plate fitted with one or more thermal probes is placed on the thermal unit of the thermal cycler and the apparatus started up according to cycles predefined by the user. The temperature data generated by the probes are then recorded, thereafter analyzed. Such a measuring system is for example marketed under the trade name CYCLERtest® by the Anachem company.

Also described is a system for evaluating the performance of PCR thermal cyclers comprising a plurality of tubes containing a water microvolume into which thermal probes are introduced, which are connected to a data acquisition means (Kim and al., 2008, BioTechniques, Vol. 44 (n° 4): 495-505).

Some of the known measuring systems at least reveal satisfactory to compensate through adjustment the occurrence of thermal variabilities in thermal cyclers.

However, there is still a need for systems measuring the accuracy and the thermal reliability of PCR thermal cyclers, that would be alternatives or improvements over known systems.

This need for systems for measuring the accuracy and the thermal reliability of PCR thermal cyclers does exist whatever the type of thermal cycler considered and both (i) for PCR thermal cyclers fitted with plates and having a thermal unit comprising a plurality of thermal elements and (ii) for PCR thermal cyclers fitted with capillary tubes wherein heating is performed through heated pulsed airflow.

### DESCRIPTION OF THE FIGURES

FIG. 1 is a diagram showing a particular embodiment of a temperature-measuring sensor used according to the invention. FIG. 1A illustrates the sensor and FIG. 1B illustrates the passage of the linking means (15) to the outside of the sensor. FIG. 1D is another diagram of the sensor illustrated on FIGS. 1An and 1B. FIG. 1C shows one embodiment of the temperature-measuring sensor, wherein the means for connecting the thermal probe to a means for receiving the signal generated by the thermal probe goes through the wall of the cover (12) of the reaction tube. FIG. 1E shows one embodiment of the temperature-measuring sensor, wherein the means for connecting the thermal probe to a means for receiving the signal generated by the thermal probe goes through the side wall (11) of the reaction tube.

FIG. 2 is a diagram showing various positioning configurations for the measuring sensors on the thermal unit of a PCR thermal cycler in accordance with various specifications, respectively.

FIG. 2A: extracted from bibliography (Schoder and al, 2003 and 2005),

FIG. 2B: extracted from an analysis report written by the Driftcon/Genotronics company,

FIG. 2C: extracted from an INRA internal publication (Hachet and al, 2005),

FIG. 2D: extracted from an analysis report written by the Applied Biosystems company,

FIG. 2E: extracted from the ISO standard n° XP CEN ISO/TS 20836 2005,

FIG. 2F: extracted from a technical report about the system marketed by the Aviso company,

FIG. 2G: extracted from a previous, non confidential but not published internal procedure,

FIG. 2H: extracted from recommendations delivered by the customer service of the Thermo Electro Corporation.

FIG. 3 is a series of layout diagrams for the measuring sensors on various thermal units of PCR thermal cyclers with 24 well-plates (3A), 30 (3B), 40 (3C), 48 (3D), 60 (3E), 96 (3F, 3G) and 384-well plates (3H, 3I) and on various thermal devices of PCR thermal cyclers with a carousel of 32 (3J), 36 (3K) and 72 (3L) wells.

FIG. 4 is a diagram showing a system for measuring the accuracy and the thermal reliability of a thermal unit in a thermal cycler.

FIGS. 5A, 5B and 5C illustrate the characteristic profiles of the temperature kinetics for the denaturation, hybridization and elongation steps. FIGS. 5A1, 5A2 and 5A3 illustrate the profiles of the temperature kinetics in an adapted thermal regulation mode of the thermal cycler. FIGS. 5B1, 5B2 and 5B3 illustrate profiles of the temperature kinetics in a thermal regulation mode of the thermal cycler comprising thermal excesses ( [symbol] overshots [symbol] ) or thermal deficits ( [symbol] undershots [symbol] ), before temperature stabilization. FIGS. 5C1, 5C2 and 5C3 illustrate profiles of the temperature kinetics in a non linear thermal regulation mode of the thermal cycler that is to say too gradual rises or decreases in temperature.

FIGS. 6 (a, b) and 7 (a, b) each illustrate the results of a biological calibration test, respectively with a thermal cycler A and with a thermal cycler B.

FIGS. 8 (a, b, c) each illustrate the results of a biological calibration test, respectively effected with a fixed hybridization temperature of 57.6° C. (8a), 58.3° C. (8b), and 59° C. (8c), on the 16 locations distributed over the unit according to scheme 3F or 3G.

FIG. 9 shows the results of three biological calibration tests, respectively at the threshold temperature Tt minus 1° C., the threshold temperature Tt minus 0.5° C. and the threshold temperature Tt.

### DETAILED DESCRIPTION OF THE INVENTION

It is provided according to the invention a method for testing the regulation of a thermal unit of a thermal cycler or an enclosure of a PCR thermal cycler thermally controlled by pulsed air, the detailed characteristics of which are specified thereafter in the present specification.

The applicant observed that with the known systems for measuring the thermal regulation of PCR thermal cyclers, of the type which comprises probes introduced into the wells of the thermal unit or of a microtest plate, a regulation variability of the thermal unit can be efficiently detected. However, thermal variabilities detected with this type of known devices do not really reflect the actual thermal variations undergone by the reaction mixtures. On the one hand, it has been observed by the applicant that these devices do measure the temperature values of the thermal unit itself and not those of the reaction mixtures. On the other hand, the substantial volume of these devices excludes any closing of the thermal cycler cover, which would cause additional temperature measurement artifacts, these additional measurement artifacts being very significant when the tested thermal cycler is fitted with a heating cover.

The applicant also observed that with the known systems provided with tubes of the type of those described by Kim and al. (2008, see Supra), the number and the arrangement of the thermal sensors in the thermal unit does not allow to detect the thermal variabilities with enough accuracy. Moreover, such systems suffered the drawback of being conceived for thermal units of one specific type, which means that as many system types are necessary as thermal units are required for performing the adjustment of the various apparatuses.

The hereabove indicated drawbacks of the known systems have been overcome according to the invention.

It is an object of the present invention to provide a method for testing the accuracy and the thermal reliability of a PCR thermal cycler, which includes (i) testing the accuracy and the thermal reliability of a plate-type PCR thermal cycler, which is fitted with a thermal unit comprising a plurality of thermally regulated elements and (ii) testing the accuracy and the thermal reliability of a capillary tube-type PCR thermal cycler, which is fitted with a thermal regulation system through heated pulsed air.

It should be noticed that the air pulsed PCR thermal cyclers comprise a thermally controlled enclosure in which are enclosed a plurality of capillary tubes (or plastic tubes depending on the apparatuses) in the hole of which the PCR amplification reaction proceeds. The thermal regulation of the enclosure in which the plurality of tubes are arranged is effected by means of a pulsed air-generating device, said device comprising at least one heating element, generally one or more electric resistances. In these PCR thermal cyclers, the pulsed airflow, which may be generated by an air blower located in said heating element, gets in contact with the heating element and is thus heated to the desired temperature. Pulsed air heated to the desired temperature is blown off to the inside of the thermally controlled enclosure where it gets in contact with the outer walls of each of the tubes wherein the PCR amplification reaction proceeds. The reaction mixture within the hole of each of the tubes is in turn brought to the desired temperature through thermal conduction. In this type of PCR thermal cycler, the tubes are generally positioned in a circular arrangement, within the thermally controlled enclosure. In general, the tubes are positioned in a circular arrangement by means of a carousel onto which the tubes are fixed. As an illustration, a capillary tube-type PCR thermal with cycler heated through pulsed air may be for example a device marketed by the Roche company under the trade name LightCycler®. As a further illustration, a PCR thermal cycler fitted with plastic tubes and heated through pulsed air may be for example a device marketed by the Qiagen company under the trade name Rotorgene®.

In the present specification, the word [symbol] thermal unit [symbol] will be preferably used to designate the heating means for the reaction mixture of a plate-type PCR thermal cycler.

In the present specification, the word [symbol] thermal enclosure [symbol] or [symbol] thermally controlled enclosure [symbol] will be preferably used to designate the heating means for the reaction mixture of a PCR thermal cycler of the capillary tube- or plastic tube-types heated through pulsed air.

Whatever the considered type of PCR thermal cycler, respectively an apparatus fitted with plates or an apparatus fitted with capillary tubes or plastic tubes heated through pulsed air, the container wherein the reaction mixture is enclosed and where a PCR amplification reaction may proceed can be commonly referred to as the [symbol] reaction mixture tube [symbol] .

It is an object of the present invention to provide a method for testing the accuracy and the thermal reliability of a thermal unit or a thermally controlled enclosure of a PCR thermal cycler, said thermal unit or said thermally controlled enclosure comprising a plurality of locations for reaction mixture tubes and a plurality of thermal regulation elements, said method comprising the following steps:
a) arranging a set of temperature-measuring sensors in locations for reaction mixture tubes selected from within said thermal unit or said thermally controlled enclosure, said temperature-measuring sensors comprising:
   (i) a reaction tube suited to the type of thermal unit tested,
   (ii) a selected volume of liquid at least partially filling said reaction tube,
   (iii) a thermal probe at least partially submerged in said liquid, and
   (iv) at least one means for linking said thermal probe to a means for receiving a signal generated by said thermal probe,
said locations in which said temperature-measuring sensors are arranged being distributed, respectively:
   across the surface of said thermal unit such that at least two temperature-measuring sensors are arranged in each of the thermal elements included in said thermal unit,
   in the enclosure thermally controlled by pulsed air at the locations provided for the PCR tubes or capillary tubes such that at least half of or at least 20 of these locations are occupied by measuring sensors, and
b) starting the thermal cycler, under normal usage conditions, for at least one cycle programmed with temperature set values for a PCR reaction, and
c) at the same time as step b), measuring the temperature values with each of the measuring sensors, at a plurality of given moments.

Depending on the thermal unit size of the thermal cycler to be tested, said thermal unit is commonly composed of two to height thermal regulation elements controlled independently from each other. The applicant demonstrated that the adjustments of the various thermal regulation elements, within the same thermal cycler, are inaccurate, which causes a variability in the temperatures applied to the reaction mixtures, depending on the thermal regulation element in wells in which the reaction tubes are arranged.

That is the reason why, to overcome this drawback, according to the method of the invention, at least two temperature-measuring sensors are arranged in the wells of each of the thermal regulation elements constituting the thermal unit of the tested thermal cycler, which enables to obtain a more reliable and reproducible thermal regulation heterogeneity measure from which thermal cycler may suffer.

As used herein, [symbol] locations for reaction mixture tubes [symbol] is intended to mean essentially, not to say exclusively:
either the [symbol] locations [symbol] provided in the controlled thermal enclosure of a PCR thermal cycler, intended to receive tubes containing a reaction mixture for implementing a PCR reaction,
or the [symbol] locations [symbol] molded in the form of wells provided in the structure of a thermal unit of a PCR thermal cycler, intended to receive the tubes containing the reaction mixture for implementing a PCR reaction, said tubes possessing a geometry and a volume especially suited to said wells or locations.

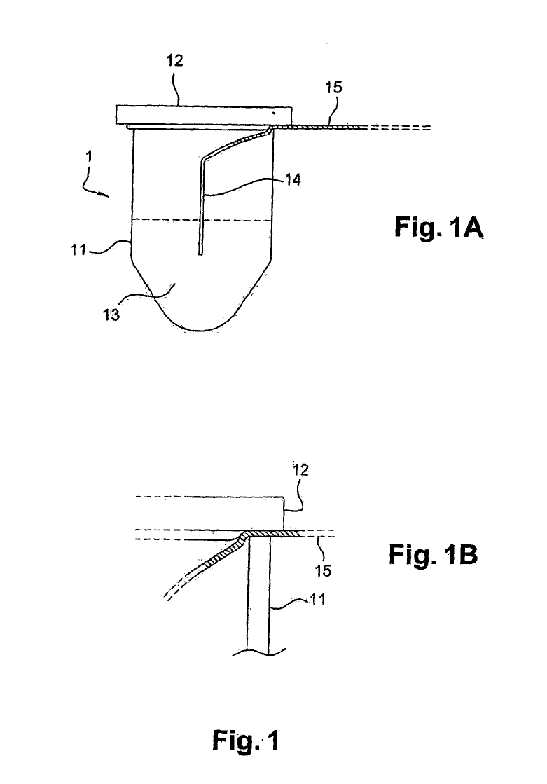

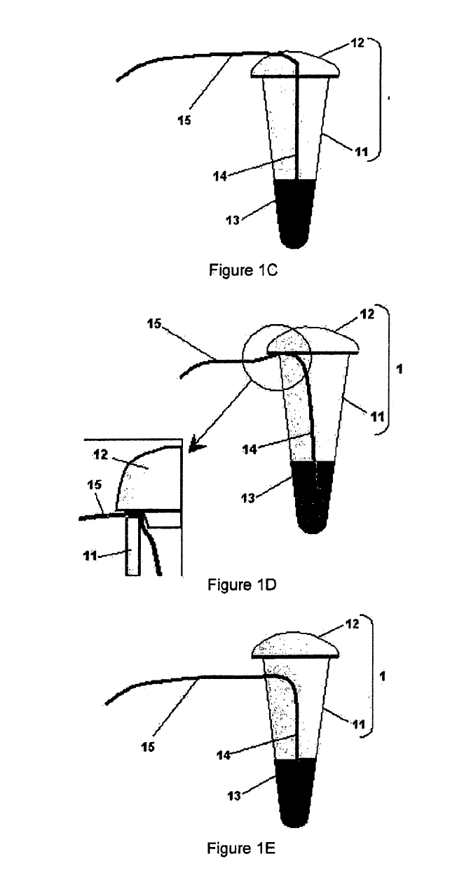

As used herein, a temperature measuring sensor is intended to mean a sensor which does possess the combined characteristics as defined hereabove. Referring to FIG. 1A, which illustrates one embodiment of a thermal sensor, said thermal sensor comprises a reaction tube (1), or a capillary tube, comprising a body (11) and a cover (12). The combined geometries of the body (11) and of the cover (12) enable their interlocking with each other. When body (11) and cover (12) are interlocked, the inside volume of the reaction tube (1) is isolated from the outer atmosphere, the junction being water-tight and gas-tight. The reaction tube (1) comprises a volume of liquid (13), wherein a thermal probe (14) is at least partially submerged, said thermal probe (14) being extended through a means (15) for linking a means for receiving the signal that might be generated by the thermal probe (14), illustrated on FIG. 1A.

As used herein, a [symbol] thermal unit [symbol] is intended to mean according to the invention the heating means for a PCR thermal cycler comprising the wells wherein the tubes are arranged which contain the reaction mixture for the PCR reactions. In general, the thermal unit consists in a heating means using the Peltier effect. Depending on the embodiments, the thermal units may comprise 24, 30, 40, 48, 60, 96 or 384 wells intended to receive the reaction mixture tubes for PCR.

As used herein, a [symbol] thermally controlled enclosure [symbol] is intended to mean according to the invention a chamber within which a carousel is housed, comprising the wells into which the tubes or capillary tubes containing the reaction mixture are to be inserted for implementing a PCR reaction. The thermal regulation of such enclosure is mediated through pulsed air. Depending on the embodiments, the thermally controlled enclosures are fitted with carousels which may comprise 32, 36, 72 or 99 wells intended to receive the reaction mixture tubes or capillary tubes for PCR.

As used herein, a [symbol] thermal regulation element [symbol] is intended to mean according to the invention any of the heating elements, generally using the Peltier effect, that are contained in a thermal unit. The thermal unit of a PCR thermal cycler comprises in most cases a plurality of thermal regulation elements. In general, each thermal regulation element is regulated independently from all the others. A thermal unit comprises commonly two to height thermal regulation elements. A great proportion of thermal cyclers are fitted with a thermal unit comprising 2, 4, 6 or 8 independent thermal regulation elements.

In step b) as used herein, [symbol] normal conditions [symbol] are intended to mean the usage conditions for which said thermal cycler has been conceived by the manufacturer. More precisely, the normal conditions include the use of the thermal cycler with [symbol] closed cover [symbol] , that is to say under the thermal regulation and insulation conditions foreseen by the manufacturer, thus avoiding any possible artifact resulting from heat losses, which can be observed with the known systems conceived to be used with the thermal cycler being under conditions of [symbol] opened cover [symbol] .

As will be illustrated in detail hereafter in the present specification, some advantageous characteristics of the measuring sensors of the invention enable to test any type of thermal cycler, including thermal cyclers fitted with means for automatically placing the reaction mixture tubes in contact with the thermal unit and wherein the thermal unit cannot be accessed by the user.

In step b), the thermal cycler is started for at least one PCR reaction cycle, that is to say for a cycle for which is normally foreseen to perform successively (i) a step for denaturing DNA, (ii) a step for hybridizing one or more nucleotide primers and (iii) a step for elongating primers which have been beforehand hybridized. Classically, the temperature set values for the steps (i), (ii) and (iii) are respectively of 95° C., 60° C. and 72° C. A variety, of other set values may be selected for each of the steps (i), (ii) and (iii), due to the fact that the major objective of the method is to determine any possible differences between the temperature set values which were initially selected for programming the thermal cycler and the temperature actual values measured by the measuring sensors. Notably the temperature set values may be fixed when programming the thermal cycler beforehand as a function of the temperatures the user whishes to use subsequently for the actual implementation of a set of PCR reaction cycles.

As will be easily understood by the person skilled in the art, the number of PCR reaction cycles programmed or effected in step b) of the method is not crucial. But the number of temperature measurement data increases as increases the number of PCR reaction cycles effected in step b). In the particular embodiments of the method wherein a plurality of cycles programmed for a PCR reaction is effected, it can be calculated for each specific moment of a cycle, and for all the cycles effected, a mean value of all the temperature values measured by each of the measuring sensors, which may increase the accuracy of the regulation test of the thermal unit.

To obtain highly reliable and reproducible regulation test results, at least three cycles are conducted in step b), more preferably at least five cycles are programmed for a PCR reaction. To obtain a highly reliable and reproducible test result, at least ten cycles programmed for a PCR reaction are conducted for example.

In step c), as used herein, a [symbol] plurality of given moments [symbol] is intended to mean that temperature measurement values are generated by each of the measuring sensors at least at three major moments within the cycle run time, respectively (i) during the DNA denaturation step, (ii) during the step for hybridizing the nucleotide primers and (iii) during the step for elongating the nucleotide primers. For each of the previously mentioned measurements, the measurement moment is preferably chosen at the moment in the step when the temperature value is normally stabilized, that is to say preferably at half time in each of these steps.

In general, the temperature is measured, in step b) of the method, at more than three given moments over the whole duration of the programmed cycles. These measurements are effected both during the previously mentioned steps and during the transition phases between each of the three previously mentioned steps, so as to check the regulation of each thermal element within the thermal unit of the thermal cycler also during the programmed temperature variation phases, which enables to make a reliability test of the thermal cycler concerning both the compliance of the temperature set values and the compliance of the set values for the starting point and the terminating point over time of the temperature programmed variations, as well as the compliance of the time duration of said programmed variations.

In practice, in step c) of the method, and for each of the sensors, at least about a hundred of temperature measurements are effected during the time of a PCR reaction cycle, so that the [symbol] plurality of given moments [symbol] generally corresponds at least to hundred. In fact, the temperature measurement rate for the sensors is usually in frequency.

In step c) of the method, a temperature value measurement is conducted in general with each of the sensors, at a frequency of at least 1 measure per second, advantageously of 2 measures per second and preferably of 4 measures per second, and in some embodiments of the method is at least of 10, 20 or 40 measures per second.

Preferably, the measuring sensors are arranged in the same thermal regulation element, in such a way that at least one first measuring sensor is positioned in a well located on one of the edges of said thermal regulation element and at least one measuring sensor is positioned in a well located in the central part of said thermal regulation element. Such an arrangement implies simultaneously (i) arranging at least two thermal probes per thermal regulation element, (ii) arranging at least one thermal probe in each corner of the thermal unit and (iii) arranging at least one thermal probe in the central part of the thermal unit.

The applicant showed that, in the known devices for measuring the regulation of the PCR thermal cyclers, the geographical distribution of the thermal probes over the thermal unit is not suitable for making measurements which results faithfully illustrate the possible heterogeneity of the accuracy and thermal reliability.

The applicant also showed that the prescriptions of the applicable standards, such as the standard ISO n° XP CEN ISO/TS 20836 2005 defining the conditions of the thermal cycler performance tests and at least as regards the localization rules of test thermal probes on the thermal units do not allow to obtain results faithfully and/or exhaustively reflecting the accuracy and the actual thermal reliability of the thermal unit tested. This standard introduces the notion of [symbol] critical space [symbol] but does not define any precise instructions for locating the thermal probes on the thermal unit. When the critical spaces are unknown, the standard recommends the random distribution of the thermal probes within the wells of the thermal unit to be tested.

The applicant further showed that, as a whole, the wells located on the periphery of the thermal units suffer from a defected thermal regulation and thus represent a [symbol] critical space [symbol] . It could be especially observed according to the invention that there is a temperature value decrease gradient for the reaction mixtures, from the center to the periphery of the thermal units. The wells located in each of the corners of the thermal unit are those for which the most important thermal regulation abnormalities could be observed.

It could also be observed according to the invention that thermal regulation heterogeneities also exist in the central part of the thermal unit, due to the fact that most of the thermal units comprise a plurality of independently thermally regulated, generally from 2 to 8 independent thermal regulation elements, for each of which a [symbol] critical space [symbol] also exists, in particular for the wells located on the periphery of said thermal regulation elements. Indeed, the wells located on the periphery of a given thermal regulation element in a thermal unit may be in fact located in the central part of said thermal unit. It is specified that, depending on the thermal cycler model considered, the geometry and the positioning of each of the thermal regulation elements constituting the thermal unit differ, so that a random thermal probe arrangement pattern on a first thermal unit having a given number of thermal regulation elements is less likely to be able to be successfully transposed for testing another thermal cycler, the thermal unit of which comprises the same number of thermal regulation elements.

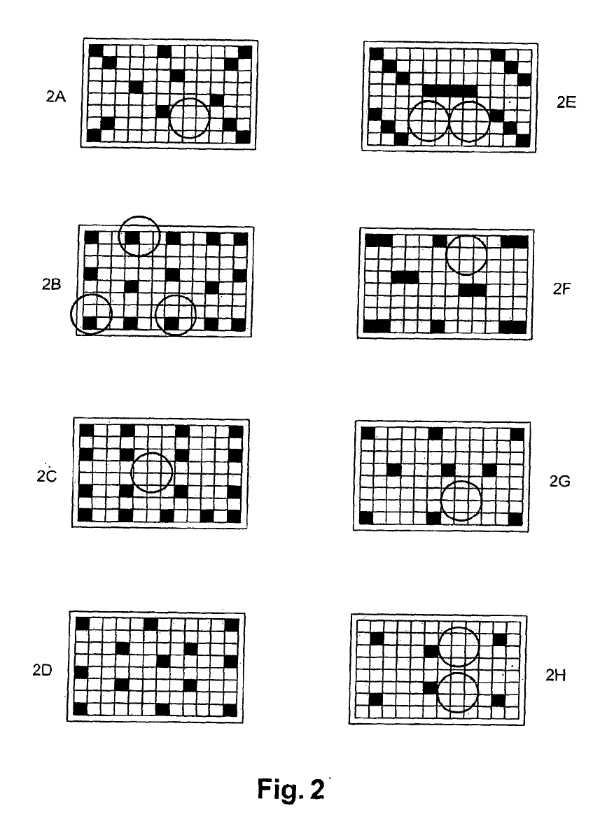

The applicant demonstrated that the instructions for positioning the thermal probes on the thermal unit of a thermal cycler to be tested, including the prescriptions included in the previously mentioned standard XP CEN ISO/TS 20836 2005, lead to a positioning of the probes that do not allow to acquire results faithfully reflecting the regulation of the thermal unit tested. By using the rules for positioning the probes stated by the previously mentioned ISO standard, with a variety of thermal cyclers fitted with a thermal unit comprising 96 wells, it was revealed that several amongst the recommended configurations for positioning the thermal probes do not allow to control all the thermal regulation elements of the thermal unit, very especially for the thermal units comprising 8 thermal regulation elements. The applicant further showed that other probe positioning configurations stated by the standard do not allow to control one or more of the critical spaces present in the thermal unit. The results of the assays performed by the applicant according to the rules included in standard XP CEN ISO/TS 20836 2005 are given on FIG. 2. FIG. 2 illustrates probe layout diagrams on thermal units provided on a variety of tested thermal cycler models. The black squares illustrate the positioning of the thermal probes. The circles illustrate, for each tested thermal cycler, the thermal regulation areas which are not controlled in the test.

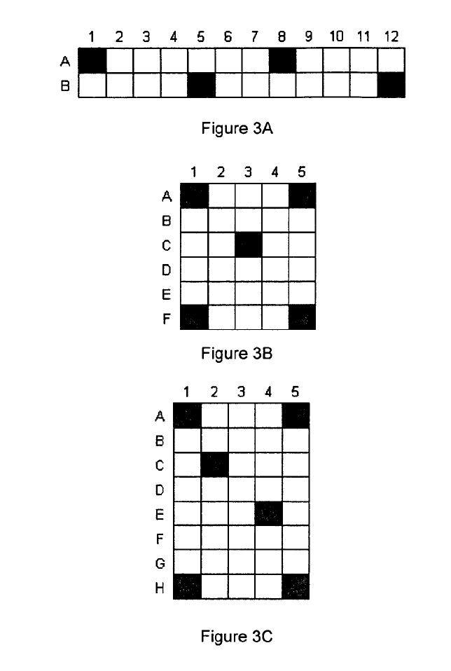

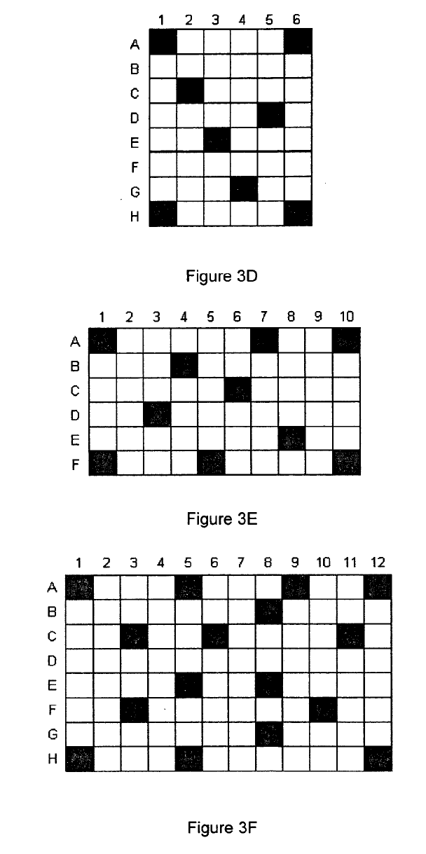

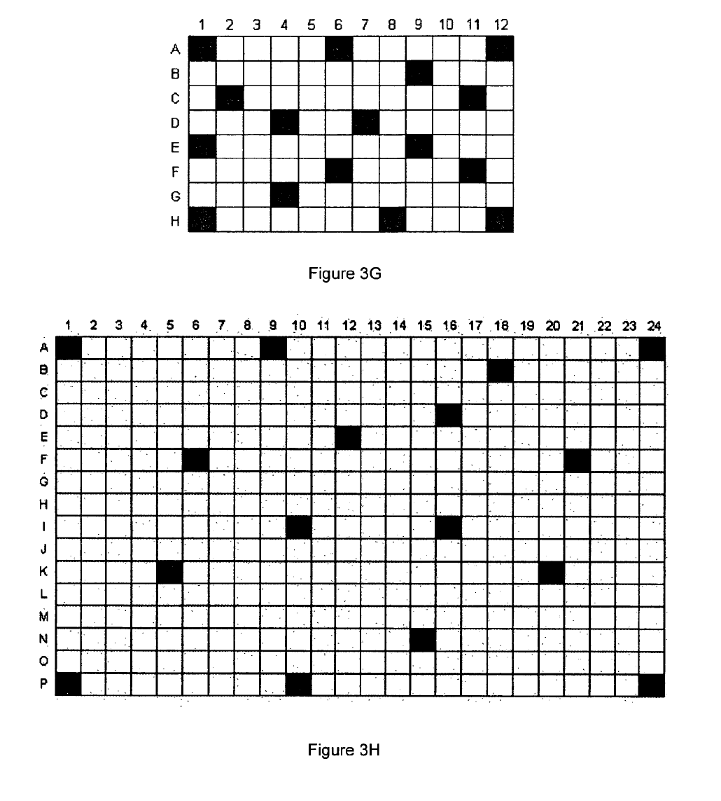

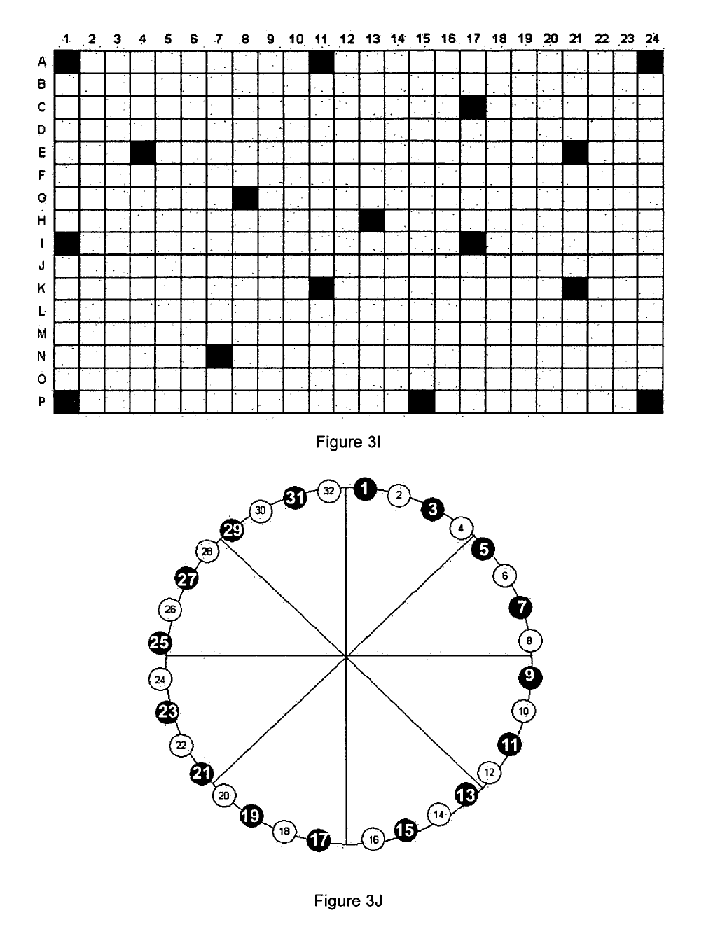

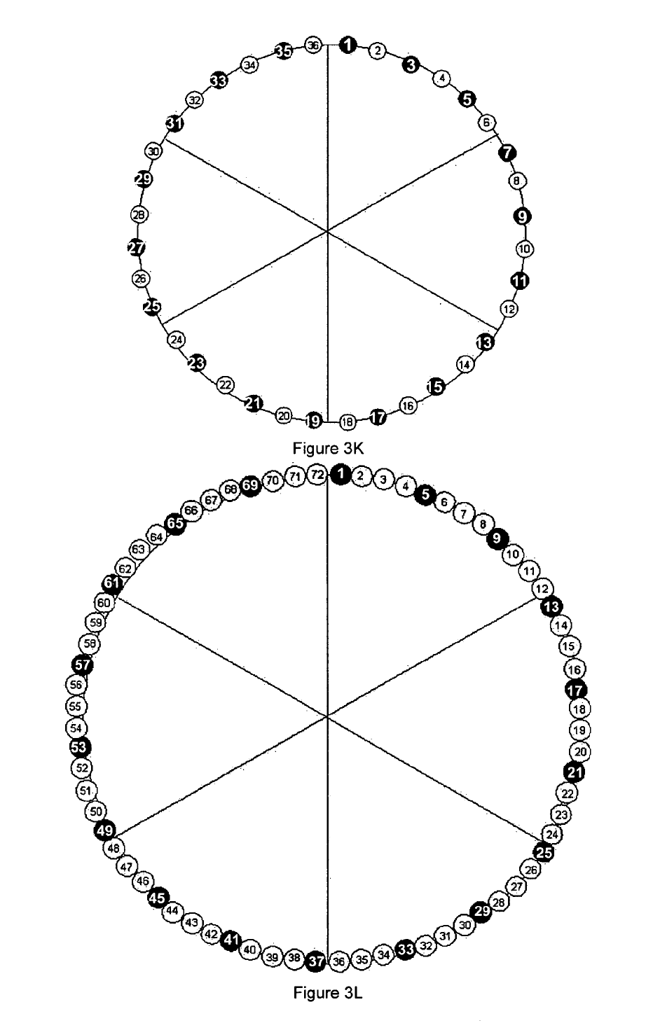

From the results obtained according to prior teachings, the applicant defined, after an intensive research, positioning configurations for the temperature-measuring sensors making it possible to test in a reliable, reproducible and exhaustive manner the accuracy and the thermal reliability of the thermal unit, whatever the type or the model of the tested thermal cycler. The preferred configurations for positioning measuring sensors according to the invention are illustrated on FIG. 3. According to the configurations illustrated on FIG. 3, at least two temperature-measuring sensors are positioned per thermal regulation element constituting the thermal unit tested and at least one temperature-measuring sensor in each of the critical space areas. FIG. 3 (3A to 3I) shows measuring sensor positioning preferred configuration for thermal units comprising 24, 30, 40, 48, 60, 96 or 384 wells. Based on the previously stated rules, and where applicable on the illustration of FIG. 3, the person skilled in the art can easily determine one or more optimal configurations for the temperature-measuring sensor positioning in any type of thermal unit, including for thermal units comprising 24, 30, 40, 48, 60, 96 or 384 wells.

Preferred configurations for temperature-measuring sensor positioning, and notably those which are illustrated on FIG. 3 (3A to 3L) represent a further object of the present invention.

In the diagrams of the thermally controlled enclosures through pulsed air, the carousels comprise 32, 36, 72 or 99 wells which by convention are circularly numbered from 1 to 32, from 1 to 36, from 1 to 72 or from 1 to 99. The temperature-measuring sensor positioning preferred configuration thus consists in the following configuration, described through the temperature-measuring sensor positioning coordinates:
32 wells (FIG. 3J): 1, 3, 5, 7, 9, 11, 13, 15, 17, 19, 21, 23, 25, 27, 29, 31 (every second well)
36 wells (FIG. 3K): 1, 3, 5, 7, 9, 11, 13, 15, 17, 19, 21, 23, 25, 27, 29, 31, 33, 35 (every second well)
72 wells (FIG. 3L): 1, 5, 9, 13, 17, 21, 25, 29, 33, 37, 41, 45, 49, 53, 57, 61, 65, 69 (every 4th well)
99 wells: 1, 5, 10, 15, 20, 25, 30, 35, 40, 45, 50, 55, 60, 65, 70, 75, 80, 85, 90, 95 (every 5th well)

In the diagrams of the units comprising 24, 30, 40, 48, 60, 96 or 384 wells (FIGS. 3A to 3I), each column comprises from 2 to 16 wells, which by convention are respectively numbered from A to P, from top to bottom of the figure. In addition each row comprises from 5 to 24 wells, which by convention are respectively numbered from 1 to 24, from the left to the right on the figure.

The temperature-measuring sensor positioning preferred configuration according to the invention on a thermal unit comprising 24, 30, 40, 48, 60, 96 or 384 wells thus consists in the following configuration, described through the temperature-measuring sensor positioning coordinates:
24 wells: 1A, 5B, 8A, 12B
30 wells: 1A, 1F, 3C, 5A, 5F
40 wells: 1A, 1H, 2C, 4E, 5A, 5H
48 wells: 1A, 1H, 2C, 3E, 4G, 5D, 6A, 6H
60 wells: 1A, 1F, 3D, 4B, 5F, 6C, 7A, 8E, 10A, 10F
96 wells: 1A, 1H, 3C, 3F, 5A, 5E, 5H, 6C, 8B, 8E, 8G, 9A, 10F, 11C, 12A, 12H (strategy 1), or 1A, 1E, 1H, 2C, 4D, 4G, 6A, 6F, 7D, 8H, 9B, 9E, 11C, 11F, 12A, 12H (strategy 2)
384 wells: 1A, 1P, 5K, 6F, 9A, 10I, 10P, 12E, 15N, 16D, 16I, 18B, 20K, 21F, 24A, 24P (strategy 1), or 1A, 1I, 1P, 4E, 7N, 8G, 11A, 11K, 13H, 15P, 17C, 17I, 21E, 21K, 24A, 24P (strategy 2).

In some embodiments of the method, at least three, four or even five measuring sensors are positioned per thermal regulation element constituting the thermal unit of the tested thermal cycler.

In some advantageous embodiments, which include the preferred configurations described hereabove, in step a) of the method, a set of 4, 5, 6, 8, 10 or 16 measuring sensors are positioned in the chosen locations of the thermal unit. The applicant demonstrated that arranging a set of 4, 5, 6, 8, 10 or 16 measuring sensors in chosen wells of the thermal unit, (i) under the previously mentioned conditions i.e. with a minimum number of sensors per thermal regulation element, and optionally (ii) under the sensor positioning conditions in each of said thermal regulation elements, represents universally applicable arrangement conditions for temperature-measuring sensors relative to the sizes of said thermal units comprising 24, 30, 40, 48, 60, 96 and 384 wells, which can be used whatever the tested thermal cycler with a similar number of wells.

Thus, according to another aspect of the method of the invention, in step a) respectively for said thermal units comprising 24, 30, 40, 48, 60, 96, 384 wells, at least 4, 5, 6, 8, 10, 16 and 16 temperature-measuring sensors are arranged, and respectively for the carousels comprising 32, 36, 72 and 99 wells of said thermally controlled enclosures through pulsed air, at least 16, 18 and 18 temperature-measuring sensors are arranged. Other advantageous arrangements of the method of the invention are described hereafter, depending especially on specific characteristics of particular embodiments of the temperature-measuring sensors used.

In some embodiments, the thermal probe of the measuring sensor consists in a thermocouple probe of a known type. Thus, the thermal probe is especially selected from the thermocouple probes of the E, J, K, N, T, R, S, B or C type and the resistance probes of the PT100 type, well known from the person skilled in the art.

Preferably, the thermal probe is a thermocouple probe of the T type made of a copper and nickel-based alloy.

According to other advantageous characteristics, the [symbol] liquid [symbol] contained in the reaction tube constituting the measuring sensor consists in a liquid which composition mimics that of a reaction mixture for carrying out a PCR reaction. In some embodiments, said liquid is distilled or demineralized water. In other embodiments, said liquid is a buffer solution of the type of those which are commonly used for carrying out PCR reactions. In still another embodiments, said liquid consists in a silicone oil, or more preferably a buffer solution of the type suited to PCR, the upper interface of which is coated with a silicone oil layer.

It has been indeed demonstrated according to the invention that the reliability and the repeatability of the method is improved when the liquid contained in the reaction tube constituting the measuring sensor of the invention has a composition or a density as close as possible to that of the reaction mixture used for carrying out a PCR reaction.

According to another preferred aspect, the [symbol] volume [symbol] of the liquid contained in the measuring sensor reaction tube is of at least 10 μL. It could be observed according to the invention that using volumes of liquid lower than 10 μL negatively influences the reliability and the reproducibility of the method.

Preferably, however, the volume of liquid contained in the reaction tube is similar to the volume of the reaction mixture which may be employed hereafter for actually carrying out PCR reactions. Depending on the circumstances, the volume of the reaction mixture employed for PCR reactions varies from 10 μL to 50 μL.

Therefore, the volume of liquid contained in the reaction tube of a measuring sensor according to the invention preferably contains from 10 μL to 50 μL of said liquid.

As used herein, a [symbol] reaction tube [symbol] constituting a temperature-measuring sensor of the invention is intended to mean any tube of a known type which is commonly used for carrying out PCR reactions, which comprises (i) a cylindrical body, generally with a tapered lower part, and (ii) a cover to isolate the inside volume of the tube from the outer atmosphere, and thus to make the tube water-tight and gas-tight. As will be detailed hereafter in the present specification, the temperature-measuring sensors used in the method can be, due to their structural characteristics, adapted to any type of thermal cycler thermal unit, whatever the form and the volume of the wells. In particular, the measuring sensors used in the method of the invention may be used with thermal units provided with wells for reaction mixture tubes having very small volumes, including tubes with volumes of 40 μL. In the embodiments of the invention where the method applies to the thermal adjustment of a PCR thermal cycler having a thermal enclosure regulated through pulsed air, a [symbol] reaction tube [symbol] includes a capillary tube or a plastic tube which is traditionally used in this type of apparatus.

Most preferably, the [symbol] means for linking [symbol] the thermal probe to the means for receiving a signal generated by said probe consists in an electric cable with a reduced diameter, for enabling the passage thereof either between the upper edge of the reaction tube and the edge of the cover in closed position (FIGS. 1A, 1D), or through the body of the tube (FIG. 1E), or through the wall of the cover (FIG. 1C).

A linking means of the electric cable type with a reduced diameter enables notably the passage thereof from the inside to the outside of the tube, without impeding the closing of said tube with the cover and therefore without altering the junction tightness between the reaction tube edges and the cover edges in a closed position.

Preferably, a linking means is used consisting in an electric cable having a diameter of at most 2 millimeters, and most preferably a diameter of at most 0.8 millimeters and at least a diameter of 0.2 millimeters.

Another advantage resulting from the use of a linking means of reduced diameter, for a measuring sensor of the invention, is the very low overall dimension of all the linking means constituting the set of measuring sensors used in the thermal regulation testing method. Such low overall dimension of all the measuring sensor linking means used contributes to the possibility of implementing the method of the invention under thermal cycler [symbol] normal usage conditions [symbol] . Indeed, the linking means low overall dimension makes it possible to run the test procedure under conditions with [symbol] closed cover [symbol] , whatever the type of tested thermal cycler, because the linking means do not hinder the normal closing of the thermal cycler. Also, the method of the invention was successfully implemented for testing thermal cyclers fitted with systems for automatically supplying the thermal unit with the reaction tubes, for which the thermal unit cannot be accessed by the user. With this type of thermal cyclers, translation of the whole linking means to the inside of the apparatus, upon actuating the supply automated system, does not hinder the displacement of the reaction tubes of the measuring sensors of the invention to the inside of the apparatus.

A further advantageous aspect resulting from the use, in a measuring sensor of the invention, of a linking means consisting in an electric cable with a reduced diameter, is that the transmission of the electric signal generated by the thermal probe, generally a thermocouple probe, does occur without any load effect. As used herein, a load effect is intended to mean that the overall weight of the thermocouples added to the reaction tubes is sufficiently low so as not to influence the temperature regulation of the thermal unit.

Furthermore, the reduced dimensions of the thermal probe/linking means assembly are adapted to the production of measuring sensors to be used for testing the accuracy and the thermal reliability of thermal units fitted with wells for tubes of very small volumes, including for 40 μL-capacity tubes. In these embodiments, a commercially available 40 μL-capacity tube can be used as the reaction tube constituting the temperature-measuring sensor.

In a temperature-measuring sensor according to the invention, the thermal probe is kept in continuous contact with the linking means, preferably with the electric cable with a reduced diameter, by means of a suitable solder, for example a metal-based solder, for example a gold or a silver solder.

In other embodiments of a sensor of the invention, the thermal probe (14) and the linking means (15) are two distinct parts of a single cable, for example a thermocouple cable of the T type based on a copper/nickel alloy.

In these embodiments, the end of the cable submerged in the reaction mixture plays the role of a thermal probe (14), the remainder of the cable, located between the end submerged in the reaction mixture and the end intended to be connected to a signal receiving means acts as a linking means (15), since the cable is electrically conductive.

Preferably, the electric cable used as a linking means does withstand a range of temperatures from 0° C. to 104° C. It results therefrom that the measuring sensor used in the method is suited to thermal regulation tests for thermal cyclers, whatever the temperature set values programmed for the implementation of a PCR reaction cycle, and in particular whatever the temperature set value programmed for the DNA denaturation step.

According to another aspect, the electric cable used as a linking means has such a mechanical resistance that it does not get damaged by the mechanical stresses to which the section of said cable interleaved between the upper edge of the reaction tube and the cover edges of said reaction tube is submitted.

The previously mentioned electric cables possess both the heat resistance and the mechanical resistance required characteristics. To optimize the mechanical resistance properties, in some embodiments, said electric cables comprise an additional covering provided onto the traditional initial cable covering. The materials used for such additional wire covering are those which have a low heat transfer coefficient and which are able to withstand temperatures of more than 105° C. The following materials are especially, not to say exclusively, concerned by the application: polypropylene, PTFE, silicone.

According to still another aspect, the linking means, by definition, has a sufficient length for effecting the transmission of the electric signal generated by the thermal probe to the signal receiving means. The length of the linking means may be easily adapted by the person skilled in the art, depending on the thermal cycler type to be tested and the spatial arrangement type of the signal receiving means, relative to said thermal cycler. Generally speaking, its is advantageous for the linking means length to be adapted for testing any thermal cycler type, in most of the spatial arrangements of the signal receiving means, relative to said thermal cycler. In practice, a linking means length of at least 50 centimeters may be chosen in most of the test situations. A length of at least 50 centimeters includes lengths of at least 60, 70, 80, 90, 100, 110, 120, 130, 140, 150, 160, 170, 180, 190 and 200 centimeters. It has been shown according to the invention that using a linking means of at least 100 centimeters is adapted to the production of measuring sensors according to the invention which may be used for test any type of PCR thermal cycler, including thermal cyclers fitted with means for automatically supplying the thermal unit with the reaction mixture tubes.

The possible passage of the linking means through the plug wall (FIG. 1C), or at the junction between the upper edges of the body of the reaction tube and the lower edges of the cover (FIG. 1A, 1D), interlocked in each other in a closed position, or through the wall of the body of said reaction tube (FIG. 1E), makes it possible to use measuring sensors which are perfectly adapted to the thermal cycler type to be tested, easily manufactured and at a reduced cost price.

Indeed, going from a first test for a thermal cycler having a thermal unit fitted with a given first well type, to a second test for a thermal cycler fitted with a given second well type that is distinct from the previous one, may require only one withdrawal operation of the thermal probe/linking means assembly from a first type of reaction tube suited to the first well type, to a second type of reaction tube suited to the given second well type, which will be filled with the chosen volume of liquid.

In other words, the method for testing the accuracy and the thermal reliability according to the invention on various thermal cycler types may be carried out without requiring a complete replacement of the material constituting a first required measuring sensor to a second required measuring sensor.

These specific advantages are obtained thanks to the measuring sensors of the invention because :
the production of a measuring sensor according to the invention does not require any specific modification of the reaction tube which is used, since the thermal probe/linking means assembly is simply introduced into said tube, and
the production of a series of measuring sensors for thermal cyclers of a distinct type does not necessarily require the use of as many thermal probe/linking means assemblies as desired measuring sensors.

For making a thermal sensor according to the invention, the thermal probe/linking means assembly is introduced into the reaction tube in such a manner that the free end of the thermal probe, which is opposed to the other end contacting the linking means, is at least partially submerged in the liquid contained in said reaction tube.

As is illustrated in the examples, the implementation of the method for testing the regulation of a thermal unit or of an enclosure thermally controlled by pulsed air of a thermal cycler may be successfully applied to determine the thermal regulation actual conditions of the apparatus. The applicant observed indeed that it does exist significant differences, sometimes highly substantial, at least concerning the accuracy and the thermal reliability, between the performance characteristics of a thermal cycler described in the technical data sheet of the manufacturer and the characteristics which are measured under usage actual conditions, by the method of the invention. Thus, the method of the invention now enables the user of a thermal cycler to precisely determine all the interesting areas of the thermal unit tested. In particular, thanks to the method of the invention, the user is from now on informed, for all the interesting areas of the thermal unit tested, and for all the time of the test comprising one or several PCR reaction cycles, about the possible temperature deviations between the set values initially programmed and the actual values. Therefore, thanks to the method of the invention, the user now has access not only to the actual temperature values in the denaturation, hybridization and elongation steps, but also to the temperature values at various moments during the temperature variation programmed phases, when successively moving, in a relatively short time of about 30 seconds to 1 minute, (i) from the step of denaturation to the step of hybridization, (ii) from the step of hybridization to the step of elongation, then (iii) from the step of elongation to the step of denaturation of the PCR reaction subsequent cycle. The conditions under which the temperature variation phases occur, which are commonly also referred to as [symbol] ramping [symbol] phases, have a significant influence on the final results. Yet, the applicant demonstrated that with many thermal cyclers, the actual temperatures of the reaction mixtures may be higher or lower than the initially programmed set values, and even, for the same [symbol] ramping [symbol] phase, (i) higher then lower than the set values or (ii) lower then higher than the set values, depending on the [symbol] ramping [symbol] phase considered, depending on the step of the PCR reaction cycle considered.

Generally speaking, the method for testing the regulation of a thermal unit or of an enclosure thermally controlled by pulsed air of a PCR thermal cycler according to the invention enables the user to know the actual performances of a PCR thermal cycler, which enables this user to adapt the programming of the apparatus, and in particular the temperature set values, and if needed also the [symbol] ramping [symbol] phase time values, in order to subsequently carry out PCR reactions under conditions that are close to the absolute chosen conditions.

Thus, the method for testing the accuracy and the thermal reliability of a PCR thermal cycler may comprise an additional step d) for determining the failures in the accuracy and the thermal reliability of the thermal unit tested, and globally of said thermal cycler.

As used herein, [symbol] determining the failures in the accuracy and thermal reliability [symbol] is intended to mean essentially a calculation enabling for a comparison between (i) set values with which the thermal cycler has been programmed and (ii) the actual operating values measured in step c) of the method.

The comparison between the set values and the actual values measured enables to calculate the [symbol] performances [symbol] of the tested thermal cycler.

The [symbol] performances [symbol] of a PCR thermal cycler include (i) the temperature accuracy, (ii) the temperature uniformity and (iii) the [symbol] ramping rate [symbol] .

The temperature accuracy consists in the average of the temperature values measured by the various measuring sensors, at a given [symbol] t [symbol] time. The temperature accuracy may be calculated based on following formula (I):

E=Tprog−Tmean (1),

with:
E which is the temperature accuracy value, for example expressed in degrees Celsius,
Tprog which is the temperature set value programmed on the thermal cycler,
Tmean which is the mean value of the temperature measured by all the measuring sensors at said time [symbol] t [symbol] , which may also be expressed in degrees Celsius,
   being understood that the mean temperature value Tmoy itself is calculated based on following formula (II):

$$Tmean = {\sum }_{i = 1}^{n} \frac{Tprobe ( i )}{n} , with \qquad ( II )$$

Tprobe(i) which is the temperature measure value measured by the measuring sensor (i), and
n which is an integer corresponding to the number of temperature-measuring sensors arranged on the thermal unit or in the enclosure thermally controlled by pulsed air.

The temperature uniformity consists in the deviation between the maximum temperature value and the minimum temperature value measured by the measuring sensors at time [symbol] t [symbol] . The temperature uniformity value may be calculated based on following formula (Ill):

U=Tmax−Tmin (III), with
U which is the temperature uniformity value, which may be expressed in degrees Celsius,
Tmax which is the maximum temperature value measured by the measuring sensors at said time [symbol] t [symbol] , and
Tmin which is the minimum temperature value measured by the measuring sensors at said time [symbol] t [symbol] .

The [symbol] ramping rate [symbol] corresponds to the time necessary for moving from a programmed temperature value to another one. The [symbol] ramping rate [symbol] may be calculated based on following formula (IV):

$$Ramping mean rate = \vert \frac{{T}_{1} - {T}_{2}}{{t}_{1} - {t}_{2}} \vert , with \text{:} \qquad ( IV )$$

[symbol] Ramping mean rate [symbol] which is the [symbol] ramping [symbol] rate value, which may be expressed in degrees Celsius per second,
T₁ which is the temperature value measured at time t₁, and
T₂ which is the value measured at time t₂

It is a further object of the invention to provide a temperature-measuring sensor for a well arranged in the thermal unit or the carousel of an enclosure thermally controlled by pulsed air of a PCR thermal cycler comprising:
(i) a reaction tube suited to the type of thermal unit tested,
(ii) a selected volume of liquid at least partially filling said reaction tube,
(iii) a thermal probe at least partially submerged in said liquid, and
(iv) at least one means for linking said thermal probe to a means for receiving a signal generated by said thermal probe.

Other advantageous characteristics of the temperature-measuring sensor according to the invention have been previously detailed in the present specification.

It is still a further object of the invention to provide a system for testing the accuracy and the thermal reliability of a PCR thermal cycler, said system comprising:
a) a plurality of temperature-measuring sensors such as previously defined, and
b) at least one means for receiving the temperature measuring signal generated by each of said sensors.

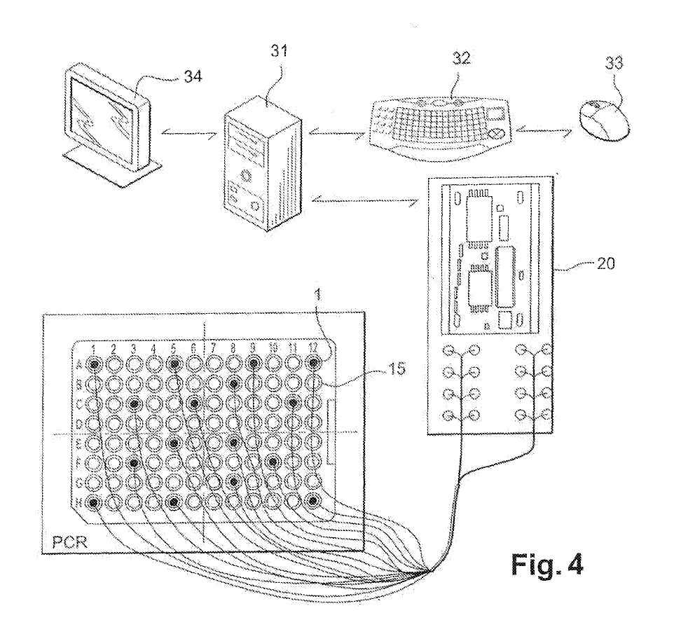

The system for testing the accuracy and the thermal reliability of a thermal unit of a thermal cycler or an enclosure of a PCR thermal cycler thermally controlled by pulsed air is described hereafter, referring to the embodiment of this system which is illustrated on FIG. 4.

Said system comprises a plurality of measuring sensors (1), which linking means (15) are connected, on their end opposed to the thermal probe of said sensor (1), to means (20) for receiving the temperature measuring signal generated by said sensor (1).

It should be noticed that, on the diagram of FIG. 4, the measuring sensors (1) are enclosed in the various wells of a thermal unit (41) provided in the thermal cycler (40) tested. On FIG. 4, although not shown in a realistic way, it should be understood that, for the implementation of the method according to the invention, the thermal cycler (41) is under normal usage conditions, that is to say with a [symbol] closed cover [symbol] , which is made possible thanks to the reduced diameter, preferably smaller than 1 millimeter, of each of the linking means (15), and thus thanks to the very small space requirement for the set composed of the plurality of linking means (15).

The signal receiving means (20) may consist in any type of device suited to receiving an electric signal generated by a thermal probe, preferably a thermocouple probe. Preferably, a signal receiving means will be used, which comprises a module for converting the analog signal delivered by each of the thermal probes to a digital signal which may then be treated in a suitable digital computer. Preferably, a signal receiving means is used, which is adapted for treating the signal generated by each measuring sensor several times per second. A receiving means is preferably used, which is able to treat the signal received from each measuring sensor at an acquisition and treating frequency of at least 5 measuring points per second, which includes at least 10, 15, 20, 25, 30, 35, 40, 45 or at least 50 measuring points per second. By definition, a receiving means will be chosen, which is able to treat the signal delivered by all the temperature-measuring sensors used in the method. For example, an acquisition means may be used, of the NetDaq® 2640A type marketed by the Fluke company.

The signal receiving means (20) is connected to the central unit (31) of a digital computer. Said digital computer comprises, in addition to the central unit (31), at least one data entry means such as a keyboard (32) or a pointing device (33), also connected to the central unit (31).

The digital computer comprises at least one data processor and at least one data storage means.

When implementing the method for testing the accuracy and the thermal reliability of a thermal unit or of an enclosure thermally controlled by pulsed air of a PCR thermal cycler, defined in the present specification, the signals transmitted by the measuring sensors (1) to the receiving means (20), if they are analog signals, are converted to digital signals. The thus generated digital signals are preferably stored in the memory of the digital computer. Said digital signals consist in a collection or a set of temperature values measured by each of the measuring sensors (1) at various times [symbol] t [symbol] . The data set may therefore come as bidimensional templates, wherein, with each stored temperature value is associated the reference of the measuring sensor (1) which did generate said value and the time [symbol] t [symbol] at which said value was generated.

The hereabove measurement data may subsequently be treated in the digital computer. For example, the memory of the digital computer may be loaded with a computer program containing a set of logic instructions for calculating the performances of the tested thermal cycler, for example respectively the values (i) of temperature accuracy, (ii) of temperature uniformity and (iii) of [symbol] ramping [symbol] rate. For example (i) calculating the performance of a PCR reaction cycle may be made, based on the accuracy and temperature uniformity for a given cycle, (ii) calculating the performance may be made, based on a plurality of PCR reaction cycles, that is to say if needed on all the cycles of a PCR complete reaction, which gives an indication of the performances calculated on all the cycles applied and on the number of measuring sensors (1) used, or even (iii) calculating the performance according to areas may be made, which consists in calculating temperature accuracy and temperature uniformity values from the temperature measurement data generated by a given measuring sensor (1), on a cycle, a plurality of cycles, or all the cycles included in a PCR reaction. This calculation enables to optimally evaluate the possible thermal regulation heterogeneity of a thermal unit or of an enclosure thermally controlled by pulsed air.

A computer program comprising a set of instructions for calculating one or several parameters for accuracy and thermal reliability performances of a thermal unit of a thermal to cycler or an enclosure of a PCR thermal cycler thermally controlled by pulsed air may be conceived for example by writing treatment macrocommands from the measurement data stored in the digital file of an usual spreadsheet program.

Preferably, the performances of the tested thermal cycler are characterized for each temperature-holding period.

Thus, thanks to the method of the invention, the users from now on can control in a very accurate way the thermal regulation conditions of the thermal cyclers and check for the stability of said thermal regulation conditions, over time.

Furthermore, the user, which now very precisely knows the temperature drift values of a given thermal cycler, as compared to the set values, is now capable of compensating for the temperature drifts measured with the method and the measuring sensors of the invention, for carrying out the PCR reaction under the same actual temperature conditions, or quasi the same, as those desired.

The method for physically testing the regulation of a thermal unit of a thermal cycler or an enclosure of a PCR thermal cycler thermally controlled by pulsed air may be completed or confirmed by a biological adjustment test, well known from the person skilled in the art.

The present invention also relates to a method for testing the accuracy and the thermal reliability of a PCR thermal cycler, comprising the following steps:
a) carrying out a physical calibration test method such as previously defined in the present specification, and
b) carrying out a biological adjustment test, using a plurality of primer pairs each having a known and distinct optimal hybridization temperature.

For the needs of the present invention, it has been specifically developed a biological adjustment test, intended to be used together with the physical adjustment test described hereabove, or to be used separately from the physical calibration test.

The biological adjustment test of the invention in its principle consists, first of all, in determining a threshold temperature for its use on a given PCR thermal cycler, then in periodically using said test, at the threshold temperature determined for said thermal cycler, so as to check the regulation of the thermal unit, on the whole thermal unit.

The threshold temperature to be determined, which characteristics are detailed hereunder, s in a primer hybridization threshold temperature, in a PCR reaction comprising a plurality of PCR reaction cycles comprising each successively (i) a step of DNA denaturation, (ii) a step of nucleic primer hybridization, and (iii) a step of primer elongation.

The biological adjustment test of the invention is based on the capacity of a plurality of nucleic primer pairs to hybridize on a target DNA, each at a specific theoretical temperature [symbol] Tm [symbol] , or melting temperature, with the successive primer pairs hybridizing at melting temperatures that are about 5° C. apart.

Preferably, in the adjustment test of the invention, three or four primer pairs are used, which hybridization temperature optimal values to target DNA, in the reaction solution used, are respectively 50° C., 55° C., 60° C. and 65° C.

The first step of the adjustment method of the invention consists in determining, specifically for the tested thermal cycler, a [symbol] threshold temperature [symbol] value, which is the hybridization temperature value measured by the apparatus at which can be detected, from the target DNA, the production of amplicons with each of the primer pairs used. As a reaction mixture, a medium is used, which comprises:
a buffer solution for PCR,
a target DNA to be amplified,
three or four primer pairs with distinct optimal hybridization temperatures, and
a thermoresistant DNA polymerase, for example a Taq polymerase.

The thermal cycler to be tested is prepared by programming the desired temperatures for each of the respective steps of DNA denaturation, of nucleic primer hybridization and nucleic primer elongation.

When nucleic primers are used, which contain a fluorescent tag, as in examples 2 and 3, the value of the [symbol] threshold temperature [symbol] is the hybridization temperature value programmed in the thermal cycler, at which fluorescence is simultaneously detected for the amplicons generated through DNA elongation from each of the primer pairs used.

The occurrence of an amplification reaction with each pair of primers visible may be visualize with (i) an electrophoretic migration on gel of all the amplified DNA, and then (ii) a fluorescent detection of DNA bands corresponding to the amplicons generated by the elongation of each of the nucleic primer pairs.

Preferably, for the threshold temperature determination step, the thermal cycler is implemented so as to produce a hybridization temperature gradient between the successive wells, in order to carry out, with the reaction medium described hereabove, simultaneous amplification reactions with a range of increasing hybridization temperatures for distinct successive wells, said hybridization temperature range covering at least the middle portion of the optimal hybridization temperature range covered by all the nucleic primer pairs used, that is to say if possible within a hybridization temperature range programmed in the thermal cycler ranging from 56° C. to 60° C.

Preferably, the PCR thermal cycler is programmed so as to obtain a hybridization temperature gradient which lower bound is of at least 45° C., which includes at least 46° C., 47° C., 48° C., 49° C., 50° C., 51° C., 52° C., 53° C., 54° C., 55° C. and 56° C.

Preferably, the PCR thermal cycler is programmed so as to obtain a hybridization temperature gradient which upper bound is of at most 70° C., which includes at most 65° C., 64° C., 63° C., 62° C. and 61° C.

For example, the thermal cycler may be programmed so as to obtain a hybridization to temperature gradient ranging from 56° C. to 60° C.

Preferably, the denaturation temperature programmed in the thermal cycler is of 95° C.

Preferably, the elongation temperature programmed in the thermal cycler is of 72° C.

The [symbol] threshold temperature [symbol] value, for the tested PCR thermal cycler, is the hybridization temperature measured by the thermal cycler of the wells for which is observed the amplification of target DNA with each of the three or four primer pairs.

Preferably, for implementing the thermoregulation biological adjustment method for a PCR thermal cycler, the following primer pairs are used:
(i) one pair of primers of sequences SEQ ID N° 1 and SEQ ID N° 2, having a hybridization optimal temperature of 50° C.,
(ii) one pair of primers of sequences SEQ ID N° 3 and SEQ ID N° 4, having a hybridization optimal temperature of 55° C.,
(iii) one pair of primers of sequences SEQ ID N° 5 and SEQ ID N° 6, having a hybridization optimal temperature of 60° C.,
(iv) one pair of primers of sequences SEQ ID N° 7 and SEQ ID N° 8, having a hybridization optimal temperature of 65° C.

The pair of primers of sequences SEQ ID N° 7 and SEQ ID N° 8 has been specifically developed for the amplification of a 483 base-pair fragment on the same gene of human DNA, in particular of human placental DNA, with a very high reproducibility. The pair of primers of sequences SEQ ID N° 7 and SEQ ID N° 8 represents an object of the present invention, as well as compositions containing the same, in particular compositions intended to be used in a biological adjustment test for determining the accuracy and the thermal reliability of a PCR thermal cycler.

Preferably, for implementing the thermoregulation biological adjustment method for a PCR thermal cycler, human placental target DNA is used. As an illustration, the human placental DNA marketed by the Sigma company, under reference n° D4642, may be used.

Thereafter, after having determined the threshold temperature for the PCR tested thermal cycler, an adjustment assay may be performed if needed, for example regularly over time or when a dysfunction is suspected, so as to check the thermal regulation of the thermal unit or of the enclosure thermally controlled by pulsed air of said apparatus.

Preferably, the accuracy and the thermal reliability of the thermal unit or of the enclosure thermally controlled by pulsed air of said thermal cycler are controlled by programming this one with the same identical temperature for all wells and placing the hereabove reaction mixture tubes in wells distributed across the surface of said thermal unit so that at least two reaction mixture tubes are enclosed in each of the thermal elements, included in said thermal unit, or for the enclosures thermally controlled by pulsed air at the locations provided for the PCR tubes or capillary tubes such that at least half of said locations are occupied by reaction mixture tubes. In general, each of the thermal elements consists, in almost all the thermal cyclers, in each of the heating elements using the Peltier effect included in the thermal unit.

It results from the previous statement that it is a further object of the present invention to provide a biological adjustment method for regulating a thermal unit of a PCR thermal cycler, comprising the following steps:
a) determining, for said thermal cycler, an adjustment threshold temperature, said adjustment threshold temperature being the hybridization temperature measured by the thermal cycler at which a human placental DNA is amplified by each of the following primer pairs: (i) a pair of primers of sequences SEQ ID N° 1 and SEQ ID N° 2, (ii) a pair of primers of sequences SEQ ID N° 3 and SEQ ID N° 4, (iii) a pair of primers of sequences SEQ ID N° 5 and SEQ ID N° 6 and (iv) more or less a pair of primers of sequences SEQ ID N° 7 and SEQ ID N° 8, in the presence of a thermo-resistant DNA polymerase, and
b) testing the regulation of the thermal unit or of the enclosure thermally controlled by pulsed air of said thermal cycler, (i) by carrying out a PCR reaction, at a programmed hybridization temperature equal to the threshold temperature determined in step a), then (ii) by checking that, at said programmed threshold temperature, said target DNA is amplified with each of the three or four primer pairs defined in step a).

Preferably, step b) is effected by arranging hereabove reaction mixture tubes in wells distributed across the surface of said thermal unit so that at least two reaction mixture tubes are enclosed in each of the thermal elements, that is to say generally each of the heating elements using the Peltier effect, included in said thermal unit.

The present invention further relates to a biological adjustment method for a PCR thermal cycler, through a regulation test of the thermal unit or of the enclosure thermally controlled by pulsed air of said thermal cycler, comprising the following steps:
a) carrying out a PCR reaction, at a programmed hybridization temperature equal to an adjustment threshold temperature of said thermal cycler, said adjustment threshold temperature being the hybridization temperature measured by the thermal cycler at which a human placental DNA is amplified by each of the following primer pairs: (i) a pair of primers of sequences SEQ ID N° 1 and SEQ ID N° 2, (ii) a pair of primers of sequences SEQ ID N° 3 and SEQ ID N° 4, (iii) a pair of primers of sequences SEQ ID N° 5 and SEQ ID N° 6 and (iv) more or less a pair of primers of sequences SEQ ID N° 7 and SEQ ID N° 8, in the presence of a thermo-resistant DNA polymerase, and
b) checking that, at said programmed threshold temperature, said target DNA is amplified with each of the four primer pairs defined in step a).

It is still a further object of the invention to provide a composition of nucleic primers for effecting a biological adjustment test of a PCR thermal cycler, comprising in a combined or separate manner the following nucleic primer pairs:
(i) a pair of primers of sequences SEQ ID N° 1 and SEQ ID N° 2, having a hybridization optimal temperature of 50° C.,
(ii) a pair of primers of sequences SEQ ID N° 3 and SEQ ID N° 4, having a hybridization optimal temperature of 55° C.,
(iii) a pair of primers of sequences SEQ ID N° 5 and SEQ ID N° 6, having a hybridization optimal temperature of 60° C., and
(iv) a pair of primers of sequences SEQ ID N° 7 and SEQ ID N° 8, having a hybridization optimal temperature of 65° C.

Preferably, in the composition hereabove, each pair of primers is included in a separate container.

Preferably, in the composition hereabove, the primers come in a freeze-dried form, and the solution of primers can be used for effecting a biological adjustment test of a PCR thermal cycler, after reconstitution by adding demineralized distilled water, or a suitable buffer solution, depending on the circumstances.

Another embodiment of a biological adjustment method according to the invention is illustrated in example 3. In this further embodiment of a biological adjustment test of the invention, the threshold temperature (Tt) is determined on the thermal cycler, from which the amplicon of promoter included and plasmid pBR322, that is to say the nucleic acid amplified thanks to the suitable primers (for example the primers of sequences SEQ ID N° 11 and SEQ ID N° 12), does not deliver any amplification signal in agarose gel. This test sensitivity is within +/−0.5° C.

The user should therefore perform temperature gradient assays for the primer hybridization step to determine this maximum threshold temperature. Thereafter, he will carry out a PCR test at three fixed primer hybridization temperatures, respectively:
temperature 1: Tt -1° C.
temperature 2: Tt -0.5° C.
temperature 3: Tt

Preferably, for the threshold temperature determination step, the thermal cycler is implemented so as to produce a hybridization temperature gradient between the successive wells, so as to cause, with the reaction medium described hereafter, simultaneous amplification reactions with a range of increasing hybridization temperatures for distinct successive wells, said hybridization temperature range covering at least the middle portion of the optimal hybridization temperature range covered by all two nucleic primer pairs used, that is to say if possible within a hybridization temperature range programmed in the thermal cycler ranging from 58° C. to 64° C.

Preferably, the PCR thermal cycler is programmed so as to obtain a hybridization temperature gradient which lower bound is of at least 55° C., which includes at least 55° C., 56° C., 57° C., 58° C., 59° C., 60° C., 61° C., 62° C., 63° C. and 64° C.

Preferably, the PCR thermal cycler is programmed so as to obtain a hybridization temperature gradient which upper bound is of at most, 70° C., which includes at most 65° C., 64° C., 63° C., 62° C. and 61° C.

For example, the thermal cycler may be programmed so as to obtain a hybridization temperature gradient ranging from 58° C. to 64° C.

Preferably, the denaturation temperature programmed in the thermal cycler is of 95° C. Preferably, the elongation temperature programmed in the thermal cycler is of 72° C.

The [symbol] threshold temperature [symbol] value, for the tested PCR thermal cycler, is the hybridization temperature measured by the thermal cycler of the wells for which a lack of amplification of target DNA pBR322 is observed with the pair of primers pBR322F01 and pBR322R03.

Preferably, for implementing the thermoregulation biological adjustment method for a PCR thermal cycler, the following primer pairs are used:
(v) one pair of primers of sequences SEQ ID N° 9 and SEQ ID N° 10, having a hybridization optimal temperature of 60° C.,
(vi) one pair of primers of sequences SEQ ID N° 11 and SEQ ID N° 12, having a hybridization maximum temperature 0.5° C. lower than the threshold temperature (Tt) from which the amplicon of promoter pBR322 does not deliver any amplification signal in agarose gel.

Thus, the present invention further relates to a biological adjustment method of the accuracy and the thermal reliability of the thermal unit or of a thermally controlled enclosure of a PCR thermal cycler, comprising the following steps:
a) determining, for said thermal cycler, an adjustment threshold temperature (Tt), said adjustment threshold temperature (Tt) being the common temperature value at which the conditions (i) and (ii) hereafter are satisfied:
   (i) the hybridization temperature measured by the thermal cycler, at which circular plasmid DNA ( [symbol] target DNA [symbol] ) is amplified by the pair of primers of sequences SEQ ID N° 9 and SEQ ID N° 10, in the presence of a thermo-resistant DNA polymerase, and
(ii) the hybridization maximum temperature measured by the thermal cycler, at which circular plasmid DNA ( [symbol] target DNA [symbol] ) is not amplified by the pair of primers of sequences SEQ ID N° 11 and SEQ ID N° 12, in the presence of a thermo-resistant DNA polymerase, and
b) testing the accuracy and the thermal reliability of the thermal unit or of the thermally controlled enclosure of said thermal cycler, by carrying out the following steps:
   b1) effecting three PCR reactions, at programmed hybridization temperatures respectively equal to (1) the threshold temperature (Tt) determined in step a), (2) a temperature 0.5° C. (Tt−0.5° C.) lower than the threshold temperature (Tt) determined in step a), and (3) a temperature 1° C. (Tt−1° C.) lower than the threshold temperature determined in step a),
   b2) checking that (1) at said temperatures (Tt-0.5° C.) and (Tt−1° C.), said target DNA is to amplified with each of the two primer pairs SEQ ID N° 9-10 and SEQ ID N° 11-12 and (2) at said threshold temperature (Tt) determined in step a), said target DNA is amplified only with the pair of primers of sequences SEQ ID N° 9 and SEQ ID N° 10.

The present invention further relates to a composition of nucleic primers for effecting a biological adjustment test of a PCR thermal cycler, comprising two primer pairs, respectively the pair of primers of sequences SEQ ID N° 9-10 and the pair of primers of sequences SEQ ID N° 11-12.

The present invention will be now further illustrated by reference to, but without limitation, the following examples.

### EXAMPLES

### Example 1

### Determination of the Temperature Kinetics Profiles

Various thermal regulation tests have been effected, with the method of the invention, on several commercially available thermal cyclers, so as to better evaluate the characteristic profiles of the PCR reaction kinetics.

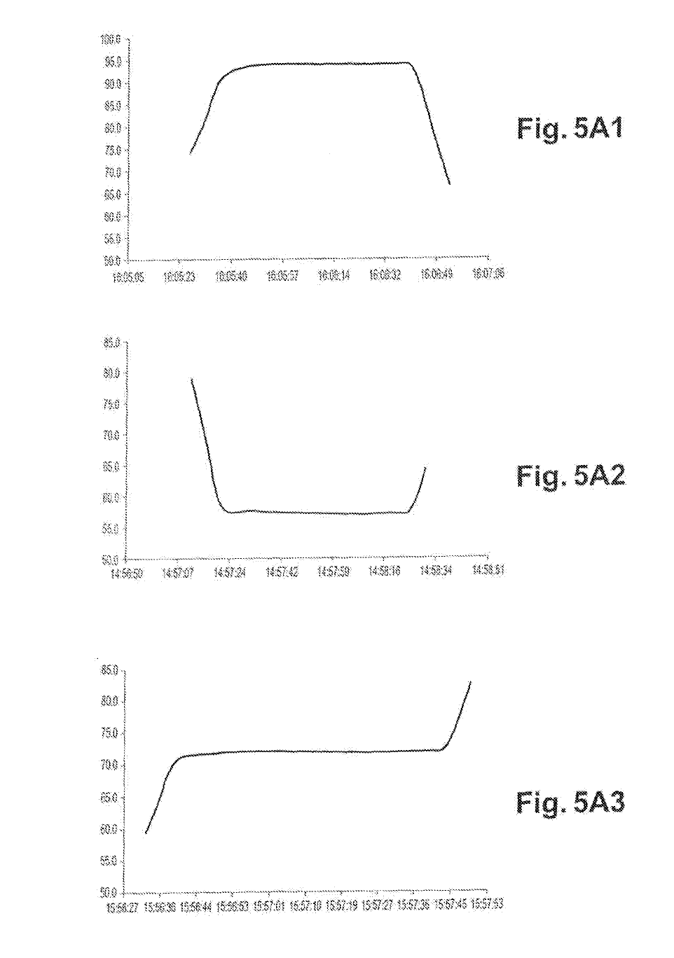

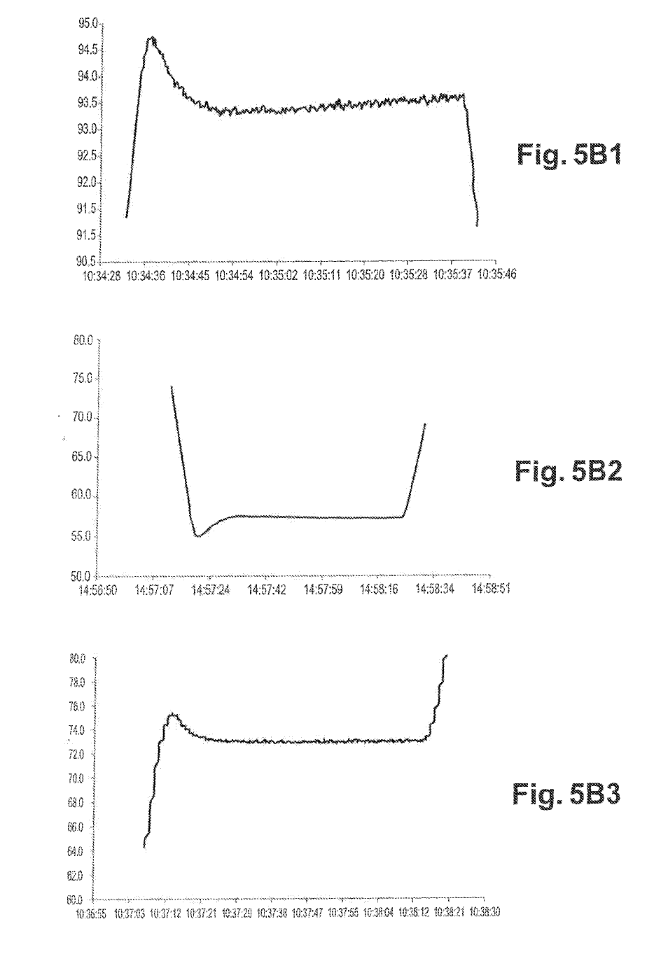

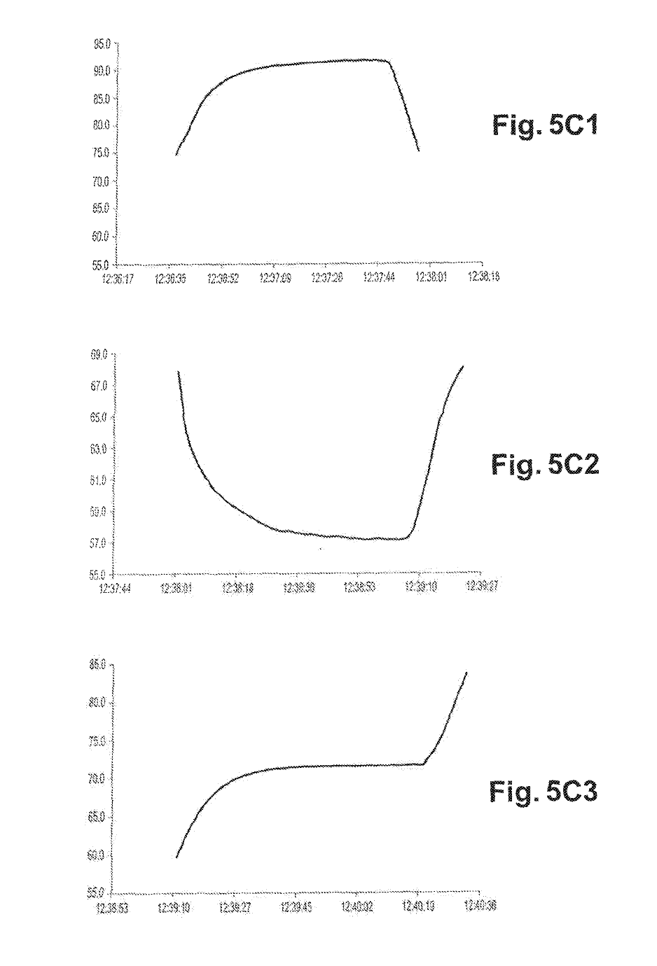

Whichever the apparatuses tested, the same profiles are generally obtained. When the regulation mode of the thermal unit or of the enclosure thermally controlled by pulsed air is correct, the temperature profiles are linear and perfectly proportioned. The beginning and the end of the plateaus can be clearly identified on the kinetics profiles of FIGS. 5A1 (denaturation step), 5A2 (hybridization step) and 5A3 (elongation step).

When the accuracy and the thermal reliability of the thermal unit or of the enclosure thermally controlled by pulsed air is heterogeneous or imprecise, a transitional step appears at the beginning of the temperature plateaus. Its duration is more or less long. This step may characterize in the form of positive or negative peaks, as compared to the mean temperature of the plateau, as illustrated on the kinetics profiles of FIGS. 5B1 (denaturation step), 5B2 (hybridization step) and 5B3 (elongation step).

The transitional step may also come as a too gradual temperature rise or decrease so that it is not possible to fix a real starting point to the plateau, as illustrated on the kinetics profiles of FIGS. 5C1 (denaturation step), 5C2 (hybridization step) and 5C3 (elongation step).

The method, the measuring sensors, and globally the regulation measuring system of a thermal unit or of an enclosure thermally controlled by pulsed air of a thermal cycler are therefore operational.

It has also been developed a digital processing program for the data collected by the receiving means of the signals delivered by the measuring sensors.

### Example 2

### Description of a Biological Calibration Test

### Strategy 2

The biological calibration test described hereafter has been effected based on the information found in the article of Yang and al (Use of multiplex polymerase chain reactions to indicate the accuracy of the annealing temperature of thermal cycling; Analytical Biochemistry, Vol. 338 (2005): 192-200).

The test targets two different genes from the same tissue. The target DNA is a human placental DNA marketed by the SIGMA company (SIGMA-Aldrich, St. Louis, Mo.; reference D4642).

The article of Yang and al (2005) describes a multiplex reaction PCR using four pairs of primers. However, the applicant observed that the fourth pair of primers described by Yang and al. (2005), referred to as [symbol] 482 [symbol] , is not functional.

The test described in the present example implements three amongst the four primer pairs described by Yang and al. (2005), respectively the primer pairs referred to as [symbol] 200 [symbol] , [symbol] 300 [symbol] and [symbol] 400 [symbol] .

The fourth pair of primers, referred to as [symbol] 480 [symbol] , has been specifically developed for the example. More precisely, the pair of primers referred to as [symbol] 480 [symbol] comprises a new specific primer, primer [symbol] DNA AS483 [symbol] of sequence SEQ ID N° 8, which enables, when used together with primer [symbol] DNA S480 [symbol] of sequence SEQ ID N° 7, to produce an amplicon of 483 base pairs.

200: fragment with an optimal hybridization
temperature: 50° C.

| | |
|---|---|
| DNA sequence S200: | |
| 5′ CACACTTCATATTTACCCAT 3′ | (SEQ ID N° 1) |
| DNA sequence AS200: | |
| 5′ TTGTTTAATAGAGACGAAGG 3′ | (SEQ ID N° 2) |

300: fragment with an optimal hybridization
temperature: 55° C.

| | |
|---|---|
| DNA sequence S300: | |
| 5′ ATGGACATTTACGGTAGTGG 3′ | (SEQ ID N° 3) |
| DNA sequence AS300: | |
| 5′ AAGTATTTCAATGCCGGTAG 3′ | (SEQ ID N° 4) |

400: fragment with an optimal hybridization
temperature: 60° C.

| | |
|---|---|
| DNA sequence S400: | |
| 5′ GCTAGCTGTAACTGGAGCCG 3′ | (SEQ ID N° 5) |
| DNA sequence AS 400: | |
| 5′ GTCTGCTGAAACTGCCAACA 3′ | (SEQ ID N° 6) |

480: fragment with an optimal hybridization
temperature: 65° C.

| | |
|---|---|
| DNA sequence S480: | |
| 5′ GGCCTGCTGAAAATGACTGA 3′ | (SEQ ID N° 7) |
| DNA sequence AS483: | |
| 5′ CAAAACAAAACAATATATACATTCCAA 3′ | (SEQ ID N° 8) |

In the specific test of the example, primer AS483 is therefore the primer complementary to S480 for the amplification of a fragment having 483 base pairs on the same gene instead of 480 base pairs, as initially predicted in the article of Yang and al. (2005).

5 to 30 ng of pure placental DNA are added per well.

### PCR End Point Tests

For amplifying several target DNAs under the priming boundary conditions and detecting the increases or decreases in temperatures, a multiplex PCR is effected using hybridization temperature gradient. The gradient consists in obtaining a hybridization temperature of the various primers either on each column, or on each line of the PCR unit (depending on the PCR apparatus models available on the market).

TABLE 1
composition of the reaction mixture for 3 or 4 bands

| Mixture for 4 bands: | | | Mixture for 3 bands: | | |
|---|---|---|---|---|---|
| | | 1 PCR | | | 1 PCR |
| | | reaction | | | reaction |
| | Cf | (μL) | | Cf | (μL) |
| buffer | 1X | 2.5 | buffer | 1X | 2 |
| (10X) | | | (10X) | | |
| H₂Oup | — | 4.83 | H₂Oup | — | 9.18 |
| 200S | 4 μM | 1 | 200S | 0.41 μM | 0.08 |
| (100 μM) | | | (104.36 μM) | | |
| 200AS | 4 μM | 1 | 200AS | 0.41 μM | 0.08 |
| (100 μM) | | | (104.36 μM) | | |
| 300S | 0.2 μM | 0.2 | 300S | 0.22 μM | 0.52 |
| (25 μM) | | | (8.46 μM) | | |
| 300AS | 0.2 μM | 0.2 | 300AS | 0.22 μM | 0.52 |
| (25 μM) | | | (8.46 μM) | | |
| 400S | 0.6 μM | 0.6 | 400S | 0.07 μM | 0.16 |
| (25 μM) | | | (8.58 μM) | | |
| 400AS | 0.6 μM | 0.6 | 400AS | 0.07 μM | 0.16 |
| (25 μM) | | | (8.58 μM) | | |
| 480S | 5 μM | 1.25 | Solution | 0.25X | 1 |
| (100 μM) | | | Q**(5X) | | |
| 483AS | 5 μM | 1.25 | MgCl₂ | 2 mM | 0.8 |
| (100 μM) | | | (50 mM) | | |
| MgCl₂ | 2 mM | 1 | dNTPs | 0.375 mM | 0.3 |
| (50 mM) | | | (25 mM) | | |
| dNTPs | 0.375 mM | 0.375 | Taq I | 1U/reaction | 0.2 |
| (25 mM) | | | (5 U/μL) | | |
| Taq I | 1U/reaction | 0.2 | TOTAL | | 15 |
| (5 U/μL) | | | ADN | to be | 5 |
| TOTAL | | 15 | template | adjusted* | |
| ADN | to be | 5 | Final | | 20 |
| template | adjusted* | | volume | | |
| Final | | 20 | | | |
| volume | | | | | |

Cf: Final concentration;
S: Sense;
AS; antisense
*the amount of target template (placental DNA) should be adjusted depending on the sensitivity of the thermal cycler used.
**the solution Q (marketed by the Qiagen company in the kit reference 203203) has been used as an adjuvant to hinder the primer fixation. It is mainly composed of betaine.

The preparation of the primer solutions is a crucial step for the experimental reproducibility over time. We strive to use pipettes suited to the volumes to be pipetted, accurate and reproducible, and having been recently metrologically controlled. A unique batch of each primer is prepared (precisely re-tested using NanoVue™ or NanoDrop™) from which aliquots are collected. Each aliquot taken from the initial batch is used for a single use. The is primer final concentrations have been established after having studied and compared the various concentrations in MgCl₂, in primers as well as in various Taq polymerases.

The PCR program used is described in Table 2 hereunder:

TABLE 2
parameters for the PCR reaction

| | | | |
|---|---|---|---|
| gradient | 95° C. | 5′ | x 1 |
| | 95° C. | 30″ | x 30 |
| | 57 to 63° C. | 30″ | |
| | 72° C. | 30″ | |
| | 72° C. | 5′ | x 1 |

First generation thermal cyclers (before 2006) have a maximum [symbol] ramping [symbol] temperature of 3° C./sec, while those of the second generation (after 2006) have a maximum ramping temperature of 4° C./sec.
However, for the apparatuses not comprising any gradient option, the PCR reaction will be performed a first time at both theoretical temperatures flanking the threshold temperature, then at the control threshold temperature where the 3 or 4 bands are present.

TABLE 3
Example of a diagram for a gradient unit:

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

3.1 Thermal cycler A

| | | | | | |
|---|---|---|---|---|---|
| A | Plac | | Plac | Plac | 60° C. |
| | D10 | | D10 | D10 | |
| B | Plac | NTC 1 | Plac | Plac | 59.8 |
| | D10 | | D10 | D10 | |
| C | Plac | | Plac | Plac | 59.5 |
| | D10 | | D10 | D10 | |
| D | Plac | | Plac | Plac | 58.9 |
| | D10 | | D10 | D10 | |
| E | Plac | | Plac | Plac | 58.2 |
| | D10 | | D10 | D10 | |
| F | Plac | | Plac | Plac | 57.6 |
| | D10 | | D10 | D10 | |
| G | Plac | | Plac | Plac | 57.2 |
| | D10 | | D10 | D10 | |
| H | Plac | NTC 2 | Plac | Plac | 57 |
| | D10 | | D10 | D10 | |

3.2. Thermal cycler B

| | | | | | | | | |
|---|---|---|---|---|---|---|---|---|
| A | Plac | Plac | Plac | Plac | Plac | Plac | Plac | Plac D10 |
| | D10 | D10 | D10 | D10 | D10 | D10 | D10 | |

| B | NTC 1 | | | | | | | NTC 2 |
|---|---|---|---|---|---|---|---|---|
| C | | | | | | | | |
| D | Plac | Plac | Plac | Plac | Plac | Plac | Plac | Plac D10 |
| | D10 | D10 | D10 | D10 | D10 | D10 | D10 | |
| E | | | | | | | | |
| F | | | | | | | | |
| G | Plac | Plac | Plac | Plac | Plac | Plac | Plac | Plac D10 |
| | D10 | D10 | D10 | D10 | D10 | D10 | D10 | |
| H | | | | | | | | |

| | | | | | | | | |
|---|---|---|---|---|---|---|---|---|
| 57° C. | 57.1 | 57.5 | 58 | 58.7 | 59.4 | 60.2 | 60.9 | 61.5 |

NTC: negative control with no DNA ( [symbol] No Template Control [symbol] ).
Plac D10: 30 ng of placental DNA diluted 10 times.

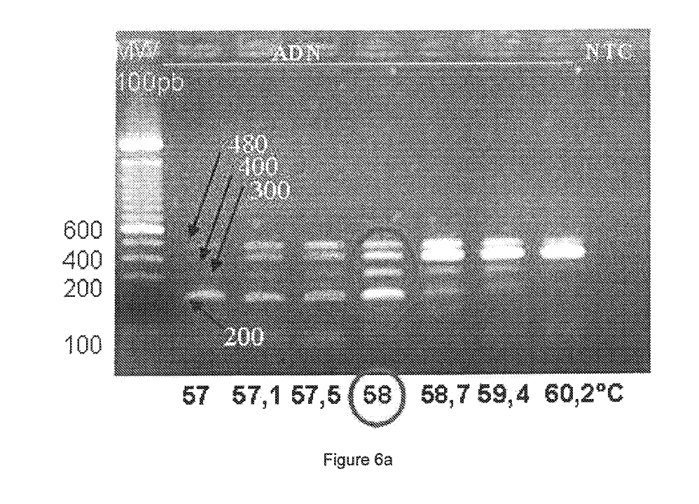

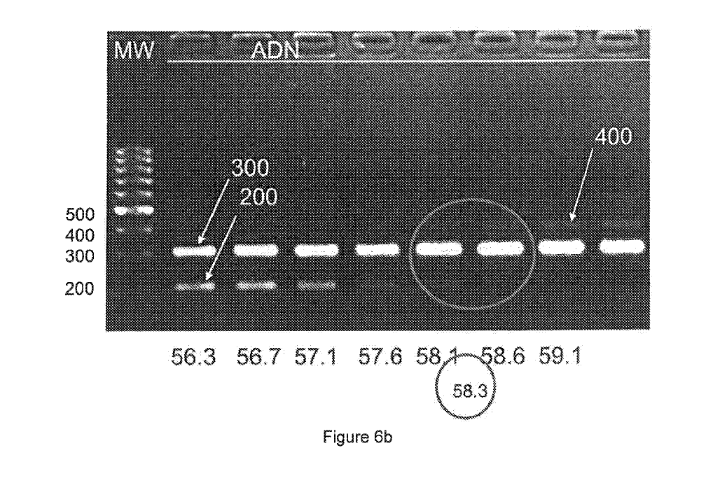

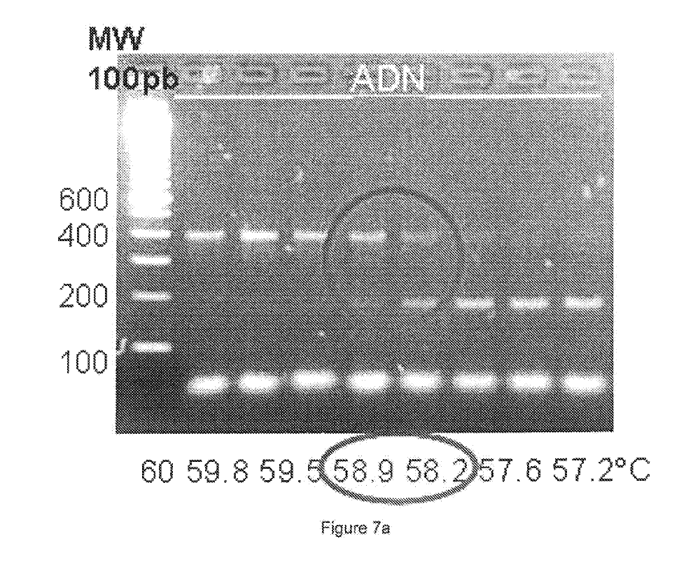

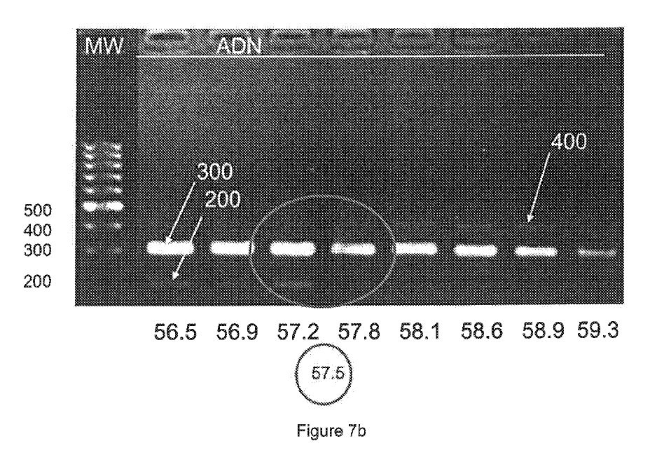

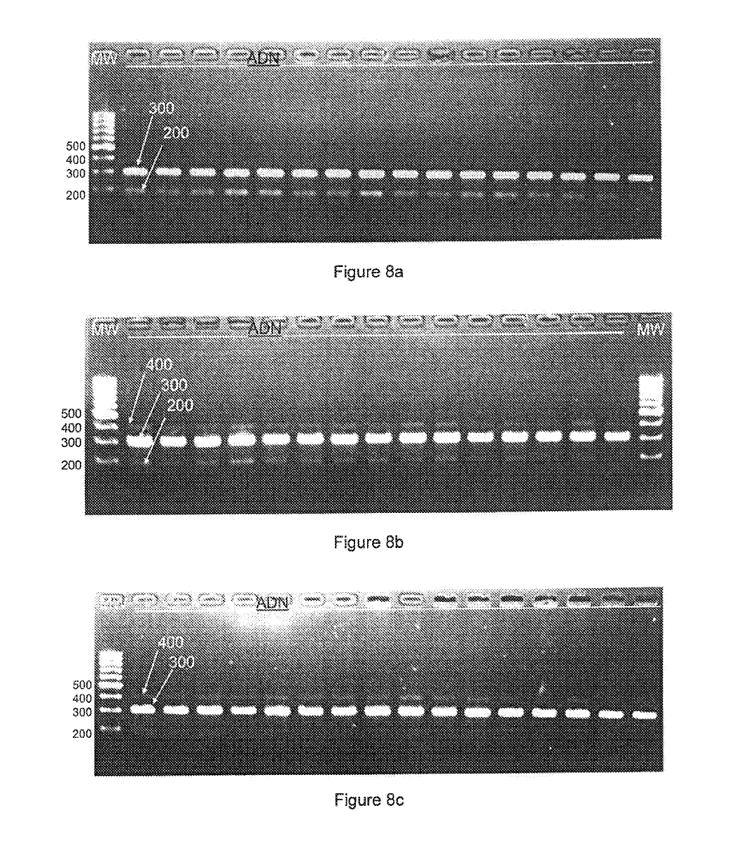

The results are given on FIGS. 6 (a, b) (thermal cycler A) and 7 (a, b) (thermal cycler B), which are amplicon migration photographs in 2% agarose gel. The electrophoretic migration is made under 120 Volts.
On FIG. 6a, the end point where the 4 bands are present with the same intensity is at 58° C. 0.7° C. is recorded before only 3 marked bands (480, 400 and 200 base pairs) and 0.7° C. after only 2 marked bands (480 and 400 bp).
This test is sensitive to an increase in temperature and to a decrease in temperature, within more or less 0.7° C.
On FIG. 6b, the end point where the 3 bands are present with the same intensity is at 58.3° C., halfway between 58.1 and 58.6° C. 0.7° C. is obtained before only 2 marked bands (400 and 200 bp) and 0.7° C. after only 2 marked bands (400 bp).
This test is sensitive to an increase in temperature and to a decrease in temperature, within more or less 0.7° C.
On FIG. 7a, the end point where the 4 bands are present with the same intensity lies between 58.9 and 58.2° C. 0.7° C. is obtained before only 1 marked band (400 bp) and 0.7° C. after only 1 marked band (200 bp). The band with 300 base pairs is very blurred and the one with 480 base pairs does not appear.
On FIG. 7b, the end point where the 3 bands are present with the same intensity lies between 57.2 and 57.8° C. 0.6° C. is obtained before 2 marked bands (200 and 300 bp) and 0.6° C. after 2 marked bands (300 and 400 bp). The 3 expected bands are present indeed.
This test differs from one apparatus to another and from one manufacturer to another. Basing on the reaction mixture described hereabove, the user should establish a control chart for each apparatus to control and thus determine the threshold temperature of the apparatus at which at least 2 amongst the 4 bands can be observed.
The development of such a control chart implies first adjusting the ramping rate of the apparatuses, then adjusting the concentration of DNA template if necessary, given that the reaction mixture is optimized to have a good balance between the various targets.
Once this chart has been established, and this threshold temperature determined, the user may limit himself to one PCR reaction on the whole plate at this threshold temperature, for example on a quarterly basis, or when he points out an abnormal result.
The gradient is only required for the test development.
This test has been effected at a fixed temperature on the three-band model on an apparatus according to the diagram for positioning the 16 points on the unit. On FIG. 8a, the selected temperature of 57.6° C. precedes the threshold temperature and only 2 bands can be observed (200 and 300).
On FIG. 8b, the selected temperature of 58.3° C. (between 58.1 and 58.6° C.) is the threshold temperature and 3 bands can indeed be observed (200, 300 and 400).
On FIG. 8c, the selected temperature of 59° C. is the temperature that immediately follows the threshold and only 2 bands can be observed (300 and 400).

### Example 3

### Description of Another Biological Calibration Test

### Strategy 3

### 1. Template

Commercial template: Gateway® pDEST™14 Vector from Invitrogen
Catalogue reference: 11801-016
Circular plasmid with 6422 base pairs dosed at 6 μg and with a known sequence.
2. Primers of the Positive Internal Control which Validates the Good Behavior of the PCR Reaction Whatever the Temperature Used Between 50 and 65° C.
The target is the sequence of the AMP gene (ampicilline) of vector pDEST14 (gene size=861 bp)
The size of the amplicon obtained is 78 bp.
The oligos drawn on the sequence of the AMP gene used are as follows:
Oligo code AMPF01: 5′ GA TAAN ATC TGG AGC CGG TGA 3′ (SEQ ID N° 9) with a simple desalting during the oligo production by the supplier.
Oligo code AMPRO1: 5′ GA TAC GGG AGG GCT TAC CAT 3′ (SEQ ID N° 10) with a simple desalting during the oligo production by the supplier.
3. Primers of the Biological Test which Enable to Detect a 0.5° C. Temperature Deviation During the Primer Hybridization Step on the Template
The target is the sequence of the promoter pBR322 of vector pDEST14 (promoter size=674 bp).
The size of the amplicon obtained when the following primers bind is 198 bp.
Oligo code pBR322F01: 5′ CC GGA TCAN AGA GCT ACC AAC 3′ (SEQ ID N° 11) with a simple desalting during the oligo production by the supplier.
Oligo code pBR322R03: 5′ CAN ACC CGG TAA GAC ACG ACC 3′ (SEQ ID N° 12) with a purification of HPLC type during the oligo production by the supplier.

Primer pBR322R03 has a mismatch on the oilgo's last base in position 3′. This mismatch creates a binding instability for this primer on the sequence target as a function of the chemical and thermal conditions of the PCR reaction.

The method of this test consists in determining on the thermal cycler the threshold temperature (Tt) from which the amplicon of promoter pBR322 does not deliver any amplification signal in agarose gel. This test is sensitive within more or less 0.5° C.

The user therefore will have to perform temperature gradient assays for the primer binding step to determine this maximum threshold temperature. Thereafter, he will make a PCR test at three fixed primer hybridization temperatures:
temperature 1: Tt−1° C.
temperature 2: Tt−0.5° C.
temperature 3: Tt

Preferably, for the threshold temperature determination step, the thermal cycler will be implemented so as to produce a hybridization temperature gradient between the successive wells, so as to cause, with the reaction medium described hereafter, simultaneous amplification reactions with a range of increasing hybridization temperatures for distinct successive wells, said hybridization temperature range covering at least the middle portion of the optimal hybridization temperature range covered by all two nucleic primer pairs used, that is to say if possible within a hybridization temperature range programmed in the thermal cycler ranging from 58° C. to 64° C.

Preferably, the PCR thermal cycler is programmed so as to obtain a hybridization temperature gradient which lower bound is of at least 55° C., which includes at least 55° C., 56° C., 57° C., 58° C., 59° C., 60° C., 61° C., 62° C., 63° C. and 64° C.

Preferably, the PCR thermal cycler is programmed so as to obtain a hybridization temperature gradient which upper bound is of at most, 70° C., which includes at most 65° C., 64° C., 63° C., 62° C. and 61° C.

For example, the thermal cycler may be programmed so as to obtain a hybridization temperature gradient ranging from 58° C. to 64° C.

Preferably, the denaturation temperature programmed in the thermal cycler is of 95° C.

Preferably, the elongation temperature programmed in the thermal cycler is of 72° C.

The [symbol] threshold temperature [symbol] value, for the tested PCR thermal cycler, is the hybridization temperature measured by the thermal cycler of the wells for which is observed the lack of amplification of target DNA pBR322 with the pair of primers pBR322F01 and pBR322R03.

Preferably, for implementing the thermoregulation biological adjustment method for a PCR thermal cycler, the following primer pairs are used:
(vii) one pair of primers of sequences SEQ ID N° 9 and SEQ ID N° 10, having a hybridization optimal temperature of 60° C.,
(viii) one pair of primers of sequences SEQ ID N° 11 and SEQ ID N° 12, having a hybridization maximum temperature 0.5° C. lower than the threshold temperature (Tt) from which the amplicon of promoter pBR322 does not deliver any amplification signal in agarose gel.

### 4. Test Conditions

### MIX PCR

| | 1 reaction (μL) | final concentration |
|---|---|---|
| H₂Oup | 7.6 | |
| MgCl₂ Promega (25 mM) | 1.2 | 1.5 mM |
| Buffer 5× Green Promega | 4 | 1X |
| pBR322F01 (10 μM) | 0.62 | 0.312 μM |
| pBR322R03 (10 μM) | 0.62 | 0.312 μM |
| AMPF01 (10 μM) | 1.25 | 0.625 μM |
| AMPR01 (10 μM) | 1.25 | 0.625 μM |
| dNTP (25 mM) | 0.25 | 0.31 mM |
| GoTaq Flexi Promega 5 U/μL | 0.2 | 1 unit i.e. 0.0625 U/μL |
| Mixed volume | 17 | |
| DNA volume(1 pg/μL) | 3 | |
| Final volume | 20 | |

### PCR Programs

| | | | Cycle |
|---|---|---|---|
| Temperature | Step | Time | number |
| 95° C. | Initial denaturation | 2 min | |
| 95° C. | Denaturation | 1 min | 28 |
| Tt −1° C. and Tt −0.5° C. | Hybridization Primers | 1 min | |
| and Tt | | | |
| 72° C. | Elongation | 20 s | |
| 72° C. | Final elongation | 5 min | |
| 15° C. | Storage | forever | |

Tt: the threshold temperature from which the amplicon of promoter pBR322 does not deliver any amplification signal in agarose gel.

### Separation on Agarose Gel

Deposition: 10 μl of the PCR product already containing the green loading buffer
TAE 0.5X
BioRad vessel
Voltage: 80V (i.e. −5.5 V.cm⁻¹)
Time: 1h50
Agarose gel 3%

### Results

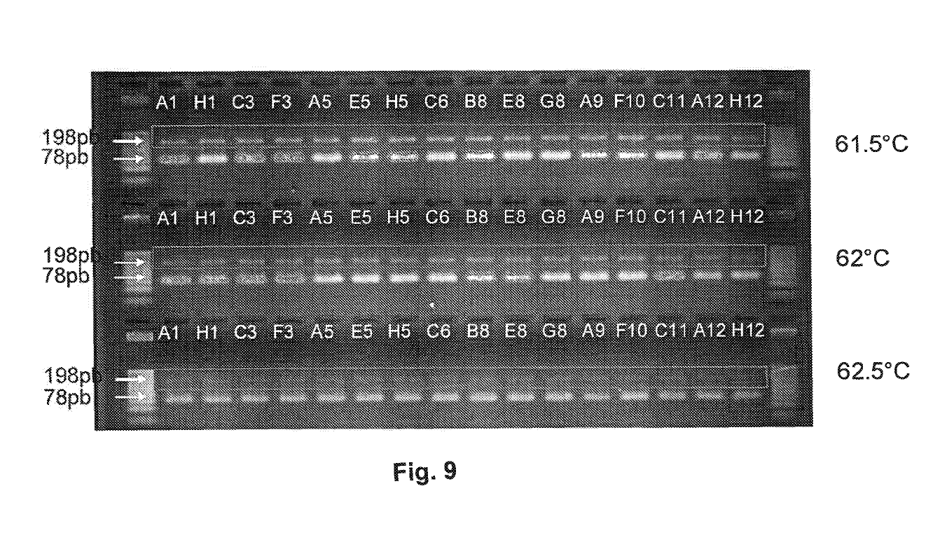

Arrangement of the samples in a 96 well-plate, according to following scheme:
Black wells: 20 μl mix PCR+DNA template, at following respective positions A1, A5, A9, A12, B8, C3, C6, C11, E5, E8, F3, F10. G8, H1, H5, H12.
White wells: 20 μl H20up, at the row and column remaining positions in the 96-well plate.
The results are illustrated on FIG. 9.
The results show that the primers of the positive internal control (SEQ ID N° 9 and 10) enable to carry out a PCR amplification reaction in the temperature range of from 50° C. to 65° C.

The results also show that the primers of the biological test (SEQ ID N° 11 and 12) enable to detect a 0.5° C. temperature deviation, as compared to the desired temperature.

1. A method for testing the accuracy and the thermal reliability of a thermal unit of a thermal cycler or a thermally controlled enclosure of a PCR thermal cycler, said thermal unit or said thermally controlled enclosure comprising a plurality of locations for reaction mixture tubes, said method comprising the following steps:
a) arranging a set of temperature-measuring sensors in locations for reaction mixture tubes selected from within said thermal unit or said thermally controlled enclosure,
said temperature-measuring sensors comprising:
(i) a reaction tube suited to the type of thermal unit tested or in the case of a thermal enclosure a capillary tube or a tube suited to the locations provided in the carousel,
(ii) a selected volume of liquid at least partially filling said reaction tube,
(iii) a thermal probe at least partially submerged in said liquid, and
(iv) at least one means for linking said thermal probe to a means for receiving a signal generated by said thermal probe,
said locations in which said temperature-measuring sensors are arranged being distributed, respectively:
across the surface of said thermal unit such that at least two temperature-measuring sensors are arranged in each of the thermal elements included in said thermal unit,
in the enclosure thermally controlled by pulsed air at the locations provided for the PCR tubes or capillary PCR tubes such that at least half of or at the most 20 of these locations are occupied by measuring sensors, and
b) starting the thermal cycler, under normal usage conditions, with heating cover being in the closed position and active), for at least one cycle programmed with temperature set values for a PCR reaction, and
c) at the same time as step b), measuring the temperature values with each of the temperature-measuring sensors, at a plurality of given moments.

2. A method according to claim 1, wherein the temperature-measuring sensors are arranged in the same thermal regulation element, in such a way that at least one first temperature-measuring sensor is positioned in a well located on one of the edges of said thermal regulation element and at least one second temperature-measuring sensor is positioned in a well located in the central part of said thermal regulation element.

3. A method according to claim 1, wherein in step a) are arranged:
at least 4, 5, 6, 8, 10, 16 or 16 temperature-measuring sensors, respectively, for thermal units comprising 24, 30, 40, 48, 60, 96 or 384 wells, or
at least 16, 18, 18 or 20 temperature-measuring sensors for thermally controlled enclosures through pulsed air comprising carousels of 32, 36, 72 or 99 wells, respectively.

4. A method according to claim 1, wherein the temperature-measuring sensors are positioned on a thermal unit of a thermal cycler with at least two temperature-measuring sensors per thermal regulation element of said unit, and so that one temperature-measuring sensor is positioned in each corner and in the center of said thermal unit.

5. A method according to claim 1, wherein the positioning coordinates of the temperature-measuring sensors on a thermal unit comprising 24, 30, 40, 48, 60, 96 or 384 wells comprising from 5 to 24 columns numbered from 1 to 24 and from 2 to 16 rows numbered from A to P are as follows:
unit of 24 wells: 1A, 5B, 8A, 12B;
unit of 30 wells: 1A, 1F, 3C, 5A, 5F;
unit of 40 wells: 1A, 1H, 2C, 4E, 5A, 5H;
unit of 48 wells: 1A, 1H, 2C, 3E, 4G, 5D, 6A, 6H;
unit of 60 wells: 1A, 1F, 3D, 4B, 5F, 6C, 7A, 8E, 10A, 10F;
unit of 96 wells: 1A, 1H, 3C, 3F, 5A, 5E, 5H, 6C, 8B, 8E, 8G, 9A, 10F, 11C, 12A, 12H (strategy 1), or 1A, 1E, 1H, 2C, 4D, 4G, 6A, 6F, 7D, 8H, 9B, 9E, 11C, 11F, 12A, 12H (strategy 2); or
unit of 384 wells: 1A, 1P, 5K, 6F, 9A, 10I, 10P, 12E, 15N, 16D, 16I, 18B, 20K, 21F, 24A, 24P (strategy 1), or 1A, 1I, 1P, 4E, 7N, 8G, 11A, 11K, 13H, 15P, 17C, 17I, 21E, 21K, 24A, 24P (strategy 2).

6. A method according to claim 1, wherein the positioning coordinates of the temperature-measuring sensors in the thermally controlled enclosures comprising carousels of 32, 36, 72, or 99 wells, respectively numbered from 1 to 32, from 1 to 36 or from 1 to 72 in a circular arrangement, are as follows:
32 wells: 1, 3, 5, 7, 9, 11, 13, 15, 17, 19, 21, 23, 25, 27, 29, 31 (every second well);
36 wells: 1, 3, 5, 7, 9, 11, 13, 15, 17, 19, 21, 23, 25, 27, 29, 31, 33, 35 (every second well);
72 wells: 1, 5, 9, 13, 17, 21, 25, 29, 33, 37, 41, 45, 49, 53, 57, 61, 65, 69 (every 4th well); or
99 wells: 1, 5, 10, 15, 20, 25, 30, 35, 40, 45, 50, 55, 60, 65, 70, 75, 80, 85, 90, 95 (every 5th well).

7. A method according to claim 1, wherein said temperature-measuring sensors comprises a thermocouple probe of the E, J, K, N, T, R, S, B or C type or a resistance probe of the PT100 type.

8. A method according to claim 1, wherein an additional covering is added to said temperature-measuring sensors, wherein the covering is of a material selected from polypropylene, polyethylene, PTFE, or silicone.

9. A method according to claim 1, wherein the means for linking the temperature-measuring sensor comprises an electric cable of a diameter smaller than 2 millimeters.

10. A method according to claim 1, which further comprises the following step:
d) determining the failures in the accuracy and the thermal reliability of said thermal unit or of the thermal enclosure controlled.

11. A method for testing the accuracy and the thermal reliability of a PCR thermal cycler, comprising the following steps:
a) carrying out a physical calibration test method according to claim 1, and
b) carrying out a biological calibration test, using a plurality of primer pairs each having a known and distinct optimal hybridization temperature.

12. A temperature-measuring sensor of a well in a thermal unit of a thermal cycler or a thermally controlled enclosure for PCR comprising:
(i) a reaction tube suited to the type of thermal unit tested or suited to the type of carousel of the thermal enclosure tested said reaction tube for receiving a selected volume of liquid to at least partially fill said reaction tube,
(ii) a thermal probe which is at least partially submergible in said liquid in said reaction tube, and
(iii) at least one means for linking said thermal probe to a means for receiving a signal generated by said thermal probe.

13. A system for testing the accuracy and the thermal reliability of a PCR thermal cycler, said system comprising:
a) a plurality of temperature-measuring sensors according to claim 1, and
b) at least one means for receiving the temperature measuring signal generated by each of said temperature-measuring sensors.

14. A biological adjustment method for regulating the thermal unit of a PCR thermal cycler, comprising the following steps:
a) determining, for said thermal cycler, an adjustment threshold temperature, said adjustment threshold temperature being the hybridization temperature measured by the thermal cycler at which a human placental DNA is amplified by each of the following primer pairs: (i) the pair of primers of sequences SEQ ID N° 1 and SEQ ID N° 2, (ii) the pair of primers of sequences SEQ ID N° 3 and SEQ ID N° 4, (iii) pair of primers of sequences SEQ ID N° 5 and SEQ ID N° 6 and optionally (iv) the pair of primers of sequences SEQ ID N° 7 and SEQ ID N° 8, in the presence of a thermo-resistant DNA polymerase, and
b) testing the regulation of the thermal unit of said thermal cycler, (i) by carrying out a PCR reaction, at a programmed hybridization temperature equal to the threshold temperature determined in step a), then (ii) by checking that, at said programmed threshold temperature, said target DNA is amplified with each of the three or four primer pairs defined in step a).

15. A method according to claim 14, in which step b) is effected by arranging the reaction mixture tubes in wells distributed across the surface of said thermal unit so that at least two reaction mixture tubes are arranged in each of the thermal elements included in said thermal unit.

16. A PCR thermal cycler biological adjustment method, through a regulation test of the thermal unit of said thermal cycler, comprising the following steps:
a) carrying out a PCR reaction, at a programmed hybridization temperature equal to an adjustment threshold temperature of said thermal cycler, said adjustment threshold temperature being the hybridization temperature measured by the thermal cycler at which a human placental DNA is amplified by each of the following primer pairs: (i) the pair of primers of sequences SEQ ID N° 1 and SEQ ID N° 2, (ii) the pair of primers of sequences SEQ ID N° 3 and SEQ ID N° 4, (iii) the pair of primers of sequences SEQ ID N° 5 and SEQ ID N° 6 and optionally (iv) the pair of primers of sequences SEQ ID N° 7 and SEQ ID N° 8, in the presence of a thermo-resistant DNA polymerase, and
b) checking that, at said programmed threshold temperature, said target DNA is amplified with each of the three or four primer pairs defined in step a).

17. A composition of nucleic primers for effecting a biological adjustment test of a PCR thermal cycler, comprising in a combined or separate manner the following nucleic primer pairs:
(i) the pair of primers of sequences SEQ ID N° 1 and SEQ ID N° 2, having a hybridization optimal temperature of 50° C.,
(ii) the pair of primers of sequences SEQ ID N° 3 and SEQ ID N° 4, having a hybridization optimal temperature of 55° C.,
(iii) the pair of primers of sequences SEQ ID N° 5 and SEQ ID N° 6, having a hybridization optimal temperature of 60° C., and
(iv) the pair of primers of sequences SEQ ID N° 7 and SEQ ID N° 8, having a hybridization optimal temperature of 65° C.

18. A pair of primers of sequences SEQ ID N° 7 and SEQ ID N° 8.

19. A biological method for adjusting the accuracy and the thermal reliability of the thermal unit or of a thermally controlled enclosure of a PCR thermal cycler, comprising the following steps:
a) determining, for said thermal cycler, an adjustment threshold temperature (Tt), said adjustment threshold temperature (Tt) being the common temperature value where the conditions (i) and (ii) hereafter are verified:
(i) the hybridization temperature measured by the thermal cycler, at which circular plasmid DNA ( [symbol] target DNA [symbol] ) is amplified by the pair of primers of sequences SEQ ID N° 9 and SEQ ID N° 10, in the presence of a thermo-resistant DNA polymerase, and
(ii) the hybridization maximum temperature measured by the thermal cycler, at which circular plasmid DNA ( [symbol] target DNA [symbol] ) is not amplified by the pair of primers of sequences SEQ ID N° 11 and SEQ ID N° 12, in the presence of a thermo-resistant DNA polymerase, and
b) testing the accuracy and the thermal reliability of the thermal unit or of the thermally controlled enclosure of said thermal cycler, by carrying out the following steps:
b1) effecting three PCR reactions, at programmed hybridization temperatures respectively equal to (1) the threshold temperature (Tt) determined in step a), (2) a temperature 0.5° C. (Tt-0.5° C.) lower than the threshold temperature (Tt) determined in step a), and (3) a temperature 1° C. (Tt-1° C.) lower than the threshold temperature determined in step a),
b2) checking that (1) at said temperatures (Tt−0.5° C.) and (Tt−1° C.), said target DNA is amplified with each of the two primer pairs of sequences SEQ ID N° 9 and SEQ ID N° 10 and of sequences SEQ ID N° 11 and SEQ ID N° 12 and (2) at said threshold temperature (Tt) determined in step a), said target DNA is amplified only with the pair of primers of sequences SEQ ID N° 9 and SEQ ID N° 10.

20. A composition of nucleic primers for effecting a biological adjustment test of a PCR thermal cycler, comprising two primer pairs, respectively the pair of primers of sequences SEQ ID N° 9 and SEQ ID N° 10 and the pair of primers of sequences SEQ ID N° 11 and SEQ ID N° 12.


SEQUENCE LISTING (12 sequences)

SEQ ID NO: 1 - DNA, 20 bp, artificial sequence
  synthetic construct primer
cacacttcatatttacccat

SEQ ID NO: 2 - DNA, 20 bp, artificial sequence
  synthetic construct primer
ttgtttaatagagacgaagg

SEQ ID NO: 3 - DNA, 20 bp, artificial sequence
  synthetic construct primer
atggacatttacggtagtgg

SEQ ID NO: 4 - DNA, 20 bp, artificial sequence
  synthetic construct primer
aagtatttcaatgccggtag

SEQ ID NO: 5 - DNA, 20 bp, artificial sequence
  synthetic construct primer
gctagctgtaactggagccg

SEQ ID NO: 6 - DNA, 20 bp, artificial sequence
  synthetic construct primer
gtctgctgaaactgccaaca

SEQ ID NO: 7 - DNA, 20 bp, artificial sequence
  synthetic construct primer
ggcctgctgaaaatgactga

SEQ ID NO: 8 - DNA, 27 bp, artificial sequence
  synthetic construct primer
caaaacaaaacaatatatacattccaa

SEQ ID NO: 9 - DNA, 20 bp, artificial sequence
  synthetic construct primer
gataaatctggagccggtga

SEQ ID NO: 10 - DNA, 20 bp, artificial sequence
  synthetic construct primer
gatacgggagggcttaccat

SEQ ID NO: 11 - DNA, 20 bp, artificial sequence
  synthetic construct primer
ccggatcaagagctaccaac

SEQ ID NO: 12 - DNA, 20 bp, artificial sequence
  synthetic construct primer
caacccggtaagacacgacc